(19) United States
(12) Patent Application Publication
(10) Pub. No.: US 2024/0325477 A1
(43) Pub. Date: Oct. 3, 2024

(54) PROTEIN HYDROLYSATE OF MORINGA PEREGRINA SEED CAKE FOR ITS APPLICATION AS A MEDICAMENT, PROCESS FOR OBTAINING SAME AND PHARMACEUTICAL AND DERMATOLOGICAL COMPOSITIONS

(71) Applicant: AGENCE FRANCAISE POUR LE DEVELOPPEMENT D'AL ULA, Paris (FR)

(72) Inventors: Elizabeth Dodinet, Saint-Laurent d'Olt (FR); Vincent Bourgeteau, Férel (FR)

(21) Appl. No.: 17/999,301

(22) PCT Filed: May 21, 2021
(86) PCT No.: PCT/EP2021/063704

§ 371 Date: Mar. 28, 2023

(30) Foreign Application Priority Data
May 21, 2020 (FR) FR2005426

(51) Int. Cl. A61K 36/185; A61K 38/01; A61P 17/00; A61P 31/14

(52) U.S. Cl. CPC A61K 36/185; A61K 38/011; A61P 17/00; A61P 31/14; A61K 2236/15; A61K 2236/17; A61K 2236/19; A61K 2236/53

(57) ABSTRACT

The invention relates to a process for obtaining a particular protein hydrolysate of Moringa peregrina seed cake. The invention also relates to a protein hydrolysate of Moringa peregrina seed cake and to its use as a medicament. Finally, the invention relates to a pharmaceutical or dermatological composition, comprising, as an active agent, an effective amount of a protein hydrolysate of Moringa peregrina seed cake for its use as a medicament for treating fibrotic diseases and treating inflammation, treating cancer, treating infectious diseases of the bacterial or viral type and treating genetic drift as well as pathologies associated with skin pigmentation.

### TECHNICAL FIELD

The invention relates to the field of pharmacy and dermatology; and more particularly to that of active ingredients in the formulation of drugs. The invention pertains to a method for obtaining a protein hydrolysate from Moringa peregrina seed cake. The invention also pertains to the protein hydrolysate from Moringa peregrina seed cake, to pharmaceutical and dermatological compositions comprising a hydrolysate of this type and intended for use in the treatment of fibrotic diseases, in the treatment of inflammation, of cancer, of infectious bacterial or viral types of diseases, as well as for the treatment of genetic drift and pathologies associated with skin pigmentation.

### BACKGROUND OF THE INVENTION

Moringaceae constitute a monogeneric family (a single genus, Moringa Adans), part of the saharo-sindian flora, constituted by between twelve and fourteen species according to the authors, distributed across east Africa and Asia. The genus is conventionally divided into 3 sections which, however, have not been confirmed to be monophyletic by phylogenetic analyses. These latter have rather highlighted clades oriented towards certain morphological features: pachycauls (bottle trees), tuberous shrubs and trees that are neither pachycauls nor tuberous shrubs (slender trees). The species Moringa peregrina (Forssk.) Fiori belongs to the third group. The few genetic studies on the genus or the family confirm the reality of the species compared with other species in this genus, in particular compared with the Indian Moringa, Moringa oleifera Lam. (see in particular the articles: OLSON, M. E., 2002, Combining Data from DNA Sequences and Morphology for a Phylogeny of Moringaceae (Brassicales), Systematic Botany 27 (1): p. 55-73; HASSANEIN, A. M. A. et al., 2018, Morphological and genetic diversity of Moringa oleifera and Moringa peregrina genotypes, Horticulture, Environment and Biotechnology 59 (2): p. 251-261). A recent article on Moringa peregrina that took samples from various locations in Saudi Arabia and used ITS markers concluded that the species is stable (ALAKLABI, A., 2015, Genetic diversity of Moringa peregrina species in Saudi Arabia with ITS sequences, Saudi Journal of Biological Sciences 22: p. 186-190) but there was a high level of intra-population genetic variation.

The species Moringa peregrina is found in rocky environments in Yemen, Oman and Saudi Arabia, in East Africa, in Sudan, in Ethiopia, Eritrea, Somalia and Djibouti. Its presence in Iran appears to be limited to south-eastern provinces, but needs to be confirmed (PROTA14=MUNYANZIZA, E. et al. Vegetable oils/oilseed plants, Moringa peregrina (Forssk.) Fiori, http://database.prota.org/protahtml/moringa peregrina_fr.htm, accessed on 23 Oct. 2019). In the Levant and in Egypt, the species found is only as rare dispersed relict plants (with the exception of a few high-altitude populations), principally in the sectors of the Sudanese region. Moringa peregrina is now also considered to be rare and endangered in Sudan and in Yemen. Compared with other species of its clade, Moringa peregrina occupies the most arid and inhospitable habitats. It is apparently more resistant to drought than Moringa oleifera, which is planted commercially on a large scale in tropical and sub-tropical zones. Recent work has shown that the size and weight of seeds will have a favorable impact on germination time and the rate and rapidity of growth of young individuals (GOMAA, N. H. et al., 2011, Seed germination, seedling traits, and seed bank of the tree Moringa peregrina (Moringaceae) in a hyper-arid environment, American Journal of Botany 98 (6): p. 1024-1030), indicating an adjustment in the allocation of resources towards the quality of the seed rather than the number of seeds, which enables Moringa peregrina to reproduce effectively in extreme abiotic environments (hyper arid environments). The seeds of Moringa peregrina have a thicker central mesotesta, in terms of cell layers, than those of Moringa oleifera.

Historically, there have been a few mentions that tend to indicate that Moringa peregrina oil was actively traded at the inception of Islam in the Al-Ula region (NASEEF, A. A. S., 1995, Al-'Ulā, A study of Cultural and Social Heritage). The oil produced from Moringa peregrina is now principally intended for domestic consumption or for local markets. In Saudi Arabia, the leaves were traditionally used as a decoction for internal use in order to treat diabetes, diseases of the colon, diseases of the eye and anemia (ABDEL-KADER et al., A survey on the traditional plants used in Al Kobah village, Saudi Pharmaceutical Journal 26 (6): p. 817-821) and as a diuretic, rubefacient and astringent (AQEEL, A. A. M. et al., 1984, Plants used in Arabian Folk medicine, Report submitted to Saudi Arabian National Centre for Science and Technology, Riyadh, Saudi Arabia). In Oman, the oil, extracted by women at the end of summer, is used against migraines, fever, burns, lacerations and fractures, constipation and stomach pain, against muscle pain and against scalp dryness (GHAZANFAR, S. A., 1994, Handbook of Arabian Medicinal Plants, 1^{st} edition, CRC Press, Boca Raton, Ann Harbor, USA; GHAZANFAR, S. A., 1998, Plants of Economic Importance, chapter 15, in GHAZANFAR, S. A. et al. (ed.) Vegetation of the Arabian Peninsula. Geobotany 25, p. 241-264, Kluwer Academic Publishers, table 11.1, p. 247 and 11.7 p. 251). It was also used in perfumed compositions (GHAZANFAR, S. A., 1998, p. 259) and in Oman and Yemen as a face lotion (GHAZANFAR, S. A. et al., 1996, Two multi-purpose seed oils from Oman. Plants for Food and Medicine. Paper presented at the joint meeting of the Society for Economic Botany and International Society for Ethnopharmacology, 1-7 Jul. 1996, London).

A certain number of extracts deriving from the Moringa oleifera seeds are known that more particularly are used in the cosmetics field. In the dermatological field, it is known from the document FR 2 776 519 that protein extracts from Moringa oleifera seeds, known for their clarifying effects on turbid water, have an emollient, physiologically conditioning, hydrating, restructuring, repair and anti-polluting effect on the skin and mucous membranes. In that document, the active principles are proteins with molecular weights comprised between 6500 and 8800 Da, which are obtained by aqueous extraction from Moringa oleifera cake.

In the pharmaceutical field, a composition is known from KR 2014 0143655 that contains a hydrosoluble extract from Moringa oleifera leaves as the active ingredient in order to treat or prevent cancer. The method for enzymatic hydrolysis on the Moringa oleifera leaf extracts can be used to isolate proteins with molecular weights comprised between 6000 and 8000 Da.

The document CN 1 0701 21 90 concerns the use of Moringa oleifera seeds in order to obtain polypeptides from the seed proteins after having extracted the oil and after carrying out an enzymatic hydrolysis using microwave radiation. The product obtained is used orally and is useful for its anti-cancer activity. That product in powder form is in fact described as having an action to reduce the side effects of chemotherapy and to increase appetite in patients presenting with liver cancer or to control the leukocyte index and body temperature.

Finally, from the document IN2009CH02906, an aqueous extract of Moringa oleifera cake comprising glucosinolate combined with cationic proteins is known that can be used as an anti-diabetic product.

All of the documents cited above concern the use of the species Moringa oleifera; none of them describes the use of extracts from the species Moringa peregrina in the pharmaceutical or dermatological fields.

In addition, the document by ABU TARBOSH et al. XP055753092, 2005 is known, which describes the extraction of a hydrolysate from shelled Moringa peregrina seeds, produced by enzymatic hydrolysis over a period of 10 hours. The aim of that extraction is to obtain a product with a high capacity for the absorption of oil and water.

An extract of Moringa peregrina oil produced from seed from Egypt using a (1/1) dichloromethane/methanol mixture exhibited an activity on three human cancer cell lines, MCF-7 (adenocarcinoma of the breast), Hep-G2 (hepatocellular carcinoma) and HCT-116 (colon carcinoma) with 1050 values of 2.92, 9.40 and 9.48 μg/mn (ABD EL BAKY et al., 2013, Characterization of Egyptian Moringa peregrina seed oil and its bioactivities, International Journal of Management Sciences and Business Research, 2 (7): p. 98-108). The authors also demonstrated a high anti-oxidizing activity by DPPH (2,2-diphenyl 1-picrylhydrazyl), ABTS (anion-scavenging capability and reducing power) and anti-proliferative assays. Because the methods for obtaining the extract differ from those of the invention, the molecules obtained have very different polarities.

ABOU-HASHEM et al (ABOU-HASHEM, M. M. M. et al., 2019, Induction of sub-G0 arrest and apoptosis by seed extract of Moringa peregrina (Forssk.) Fiori in cervical and prostate cancer cell lines, Journal of Integrative Medicine 17: p. 410-422) have also demonstrated an induction activity of the sub-G₀ arrest and apoptosis on cervical (HELA) and prostate (PC-3) cancer cell lines. The protocol involved Egyptian Moringa peregrina seeds reduced to powder and extracted with 95% ethanol, then dissolved in an aqueous solution from which three fractionated extracts were studied (fractions using petroleum ether (PE), CHCl₃ and EtOAc) as well as a hydroalcoholic extraction residue. The activity was demonstrated for the total chloroform fraction and was attributed to saturated and unsaturated fatty acids and to polyphenols without a specific study of the mechanism. Because the methods for obtaining the extract were different from those of the present invention, the active molecules obtained had very different polarities.

In view of the foregoing, a problem which the invention proposes to overcome is the development of novel products based on an extract of the species Moringa peregrina from the genus Moringa and the Moringaceae family that can be used in pharmacy or in dermatology and that are easy to use.

Thus, the Applicant has surprisingly developed a particular protein hydrolysate obtained from Moringa peregrina seed cake for its application as a drug, and in particular, for the treatment of fibrotic diseases (as an inhibitor of furin convertase), the treatment of inflammation, of cancer, of infectious diseases of the bacterial or viral type as well as the treatment of genetic drift and of pathologies associated with skin pigmentation.

The species Moringa peregrina grows in very arid climates. Thus, its ability to resist drought and to reproduce under extremophile conditions has enabled it to acquire particular unique characteristics that the Applicant has been able to identify by carrying out a specific extraction method on Moringa peregrina seed cake. The protein hydrolysate in accordance with the invention has been shown to have properties as a drug and it has been demonstrated that in particular, it has a very high furin convertase inhibiting activity.

Furin convertase (hereinafter termed furin) is a type 1 transmembrane protein with 794 amino acids expressed in different cell types. Furin is a protein which has been shown to be involved in a large number of biological processes (BRAUN E. et al., 2019, Furin-mediated protein processing in infectious diseases and cancer, Clinical & Translational Immunology https://doi.org/10.10021cti2.1073). It is particularly involved in the cicatrisation of wounds as well as in the regulation of fibrosis in the treatment of disorders in which fibrosis is a major tissue repair mechanism or when excessive fibrosis causes pathological disruption and tissue dysfunction (see the document WO 2004/09113 in this regard, which concerns the use of convertase in order to reduce cicatrisation during healing of an injury and to reduce fibrosis in the treatment of fibrotic states). The cicatrisation of wounds in an adult is a complex repair process. Wounds can involve damage, injuries or trauma to a tissue or an internal organ such as the lungs, kidneys, the heart, the intestines, the tendons or the liver. The wound healing process in tissue generally commences by a hemostatic response triggered by damage to the blood vessels of the skin. During this process, the conjunctive tissue that is formed during the healing process is often fibrous in nature and generally forms as a conjunctive tissue scar (a process known as fibrosis). Thus, fibrosis may include pulmonary fibrosis, renal fibrosis, hepatic fibrosis, skin fibrosis, ocular fibrosis, cardiac fibrosis, and other various fibrotic states. The protein hydrolysates of the invention may also be useful in the treatment of other pathological states mediated by furin including, but not limited to: hypertension, cancer, infectious diseases (bacterial and viral) and genetic disorders (for example cystic fibrosis (CF)), and neurodegenerative disorders (see the document WO 2019/215341 in this regard, which concerns novel furin convertase inhibitor compounds and pharmaceutical compositions containing them, and which cites a number of publications demonstrating the pharmaceutical activity of inhibitors; these citations are hereby incorporated into the present description by reference).

By means of an intergovernmental agreement between the government of the French Republic and the Kingdom of Saudi Arabia of Apr, 10 2018, the Applicant, Agence Française Pour Le Développement d'AlUla (AFALULA) [French Agency for AlUla] as well as the Commission Royale pour AlUIA (RCU) [Royal Commission for AlUla] instigated a joint project particularly for the development of responsible agriculture and local economy, in particular by the local production of natural products derived from indigenous plants and for the protection of the biodiversity and laws of the AlUla region of the Kingdom of Saudi Arabia. The Kingdom of Saudi Arabia has been a member of the Nagoya Protocol since Oct. 8, 2020. At the time of drafting the present patent, the rules by virtue of which the Nagoya Protocol will be integrated into the pertinent aspects of local law are under consideration. As a consequence, at this stage, the Kingdom of Saudi Arabia has no specific demands as regards this patent application and the Nagoya Protocol. Thus, as at the day of filing of the patent application, there is no requirement for a certificate of conformity regarding access to genetic resources.

### SUMMARY

In a first aspect, the invention provides a method for obtaining a protein hydrolysate from Moringa peregrina seed cake, comprising the following steps, in which:
a) unshelled mature seeds are collected from ripe Moringa peregrina fruit and are dried in order to obtain an internal moisture content of less than 8%,
b) the dried seeds are pressed in a manner such as to separate the oil from the remainder of the seed, in a manner such as to obtain the cake comprising less than 6% by weight of residual oil,
c) the cake obtained in step b) is ground,
d) the ground cake obtained in step c) is dispersed in an aqueous phase,
e) the aqueous dispersion obtained in step d) undergoes chemical proteolysis for a period of approximately 2 hours, at a pH of more than 13 and at a temperature comprised between 16° C. and 25° C.,
f) the proteolysis is neutralized in order to stabilize the protein hydrolysate obtained,
g) the protein hydrolysate is recovered by solid/liquid separation,
h) the protein hydrolysate is purified by ultrafiltration and/or nanofiltration, then optionally,
i) the protein hydrolysate obtained in step h) is lyophilized.

In a second aspect, the invention concerns a protein hydrolysate from seed cake that has not been shelled and has been harvested from ripe Moringa peregrina fruit, comprising a major fraction of amino acid derivatives, amino acids, peptides and glycopeptides for which the molecular weight P1 is comprised between 1500 Da and 5000 Da, a fraction of approximately 20% for which the molecular weight P2 is comprised between 10000 and 17000 Da and a fraction of approximately 20% for which the molecular weight P3 is approximately 23000 Da, in that it is obtained by chemical proteolysis at a pH of more than 13 for a period of approximately 2 hours at a temperature comprised between 16° C. and 25° C. and in that it is liquid and has a density of more than 1 and preferably about 1.1.

Because of its characteristics, the Moringa peregrina protein hydrolysate in accordance with the invention has never been disclosed in the genus Moringa and the Moringaceae family. It will be demonstrated that the extract from the species Moringa peregrina has a particular peptide profile that differs from other species of the genus, in particular the species Moringa oleifera, which the Applicant has been able to demonstrate.

In a third aspect, the invention concerns a protein hydrolysate from Moringa peregrina seed cake for its application as a drug.

In a fourth aspect, the invention concerns a pharmaceutical or dermatological composition comprising, as the active agent, an effective quantity of a protein hydrolysate from Moringa peregrina seed cake and a physiologically acceptable excipient.

Finally, in a fifth aspect, the invention concerns a pharmaceutical or dermatological composition intended for use in the treatment of fibrotic diseases, the treatment of inflammation, of cancer, of infectious diseases of the bacterial or viral type as well as in the treatment of genetic drift and of pathologies associated with skin pigmentation.

### BRIEF DESCRIPTION OF THE FIGURES

The invention will be better understood and other aims, details, features and advantages thereof will become more apparent from the following description of several particular embodiments of the invention, given solely by way of non-limiting illustration and with reference to the accompanying drawings.

FIG. 1 represents a diagram of infection by a SARS-COV2 (Severe Acute Respiratory Syndrome Coronavirus 2) coronavirus that demonstrates the anti-infectious and virostatic effect of the peregrina protein hydrolysate in accordance with the invention against SARS COV2 coronavirus pseudovirions.

FIG. 2 represents the inhibition diagram for furin using an extract of peregrina oil. In this figure, ** means significantly different from the “control” group (P<0.001).

FIG. 3 represents the inhibition diagram for furin using a peregrina cake extract (96° ethanol). In this figure, * means significantly different from the “control” group (P<0.001).

FIG. 4 represents the inhibition diagram for furin using a protein hydrolysate from peregrina cake in accordance with the invention. In this figure, *** means significantly different from the “control” group (P<0.001).

### DESCRIPTION OF EMBODIMENTS

In this description, unless specified othemise, it should be understood that when a range is given, it includes the upper and lower limits of said range.

In the present invention, the abbreviations below mean the following:
MTT: 3-(4,5-dimethylthiazol-2-yl)-2,5-diphenyl tetrazolium bromide (the MTT test is a rapid method for counting living cells)
SDS: Sodium Dodecyl Sulfate
PBS: Phosphate Buffer Saline
ELISA: Enzyme-Linked Immunosorbent Assay
PCR: Polymerase Chain Reaction
ANOVA: Analysis Of Variance
MSH: Melanocyte Stimulating Hormone

In the present invention:
“mainly amino acid derivatives, amino acids, peptides and glycopeptides” refers to a quantity of more than 20%, more preferably more than 30% and possibly up to approximately 40% (Weight/Weight) dry matter of amino acid derivatives, amino acids, peptides and glycopeptides, more preferably 50% by weight of dry matter.
“effective quantity” refers to the quantity of active molecules that are necessary in order to obtain the desired result, namely of guaranteeing the desired therapeutic activity.
“proteolysis” refers to the segmentation of proteins into peptides, oligopeptides and its base fragments (amino acids) and its residues by means of chemical hydrolysis.
“local administration” refers to the fact that the drug, applied directly to its point of action, exerts its pharmacological effect on the exact site of the disorder. The aim of local administration is to limit diffusion of the active principle from its administration site, enabling undesirable effects to be minimized. Cutaneous, nasal and respiratory administration, ocular administration, auricular administration, vaginal administration and buccal administration are the principal local administration modes that are employed.
“topical application” refers to the fact of applying or spreading the active principle in accordance with the invention or a composition containing it onto the surface of the skin, mucous membranes or the nails and haft.
“physiologically acceptable” in the context of topical use refers to contact with the human skin, or in the context of other modes of administration, for example oral administration or by injection into the skin, refers to zero risk of toxicity, incompatibility, instability or allergic response.
“cake” refers to the defatted seed after pressing. It is a solid residue left following extraction of the oil from the seed. It is a co-product in trituration, an oil production process. It generally represents 50% to 75% of the mass of the seeds.
“unshelled seeds” refers to the fact that the shell (pericarp) and the coat of the harvested seeds are maintained around the kernels.
“when the fruit is ripe” refers to the fact that the fruit is mature, preferably when the hull is ready to split open and it takes on a dark beige to brown color and when the lower quarter of the hull is twisted by 180°, valve opening is triggered.
“approximately” refers to a margin of plus or minus 10% to 20% with respect to the given information.
“pool of active molecules”, “active agent” and also “active principle” refer to the protein hydrolysate extracted in accordance with the method of the invention starting from Moringa peregrina seed cake. This hydrolysate is responsible for the biological activities described in the present invention.
“active agent” refers to a sufficient quantity of an extract in accordance with the invention in order to obtain the described biological activities. Depending on whether the extract is liquid or dry, or concentrated or otherwise, the quantities of active agent may vary within proportions of 0.0001% to 40% by weight with respect to the total weight of the composition.

In a first aspect, the invention concerns a method for obtaining a protein hydrolysate from Moringa peregrina seed cake, comprising the following steps in which:
a) unshelled mature seeds are collected from ripe Moringa peregrina fruit and are dried in order to obtain an internal moisture content of less than 8%,
b) the dried seeds are pressed in a manner such as to separate the oil from the remainder of the seed in a manner such as to obtain the cake comprising less than 6% by weight of residual oil,
c) the cake obtained in step b) is ground,
d) the ground cake obtained in step c) is dispersed in an aqueous phase,
e) the aqueous dispersion obtained in step d) undergoes chemical proteolysis for a period of approximately 2 hours, at a pH of more than 13 and at a temperature comprised between 16° C. and 25° C.,
f) the proteolysis is neutralized in order to stabilize the protein hydrolysate obtained,
g) the protein hydrolysate is recovered by solid/liquid separation,
h) the protein hydrolysate is purified by ultrafiltration and/or nanofiltration carried out with a cutoff threshold comprised between 100 and 25000 Da, then optionally,
i) the protein hydrolysate obtained in step h) is lyophilized.

The unshelled seeds are collected, i.e. wherein the shell is retained when the fruit is ripe and preferably when the hull is starting to split.

The seeds are dried in order to obtain an internal moisture content of less than 8% and preferably about 6%; drying will preferably be carried out on a ventilated rack shielded from the sun's rays, preferably in the shade in the open air.

Next, the dried seeds are ground extemporaneously with cold pressing, which can be used to mechanically separate the oil from the remainder of the compressed seed, namely the cake.

The cake is then ground mechanically with any type of mechanical grinder, such as a hammer mill, flail mill, knife mill, crushing/shredding mill, a ball mill or beater mill, but also with any type of cryomill.

The dispersion to form the aqueous phase in accordance with step d) and the proteolysis in accordance with step e) are advantageously carried out with continuous stirring, thereby enabling dispersion and homogenization of the solid in the liquid, therefore improving the overall surface area for exchange and, as a result, improving proteolysis.

A liquid protein hydrolysate with a density of more than 1 and preferably about 1.1 is obtained, comprising a dry matter content comprised between 10% and 15%, preferably about 12.5%, comprising between 1% and 6% of nitrogen-containing compounds, in particular volatile nitrile derivatives, in a proportion of 0.5% to 1.5%, preferably about 0.8%, as well as 20 mg/liter of polyphenols (0.002%).

In accordance with a preferred embodiment of the method in accordance with the invention, the temperature of the proteolysis of step f) is approximately 22° C.

In accordance with a preferred embodiment of the method in accordance with the invention, the solid/liquid separation of step g) is carried out using different methods such as centrifuging, draining, filtration.

In one embodiment of the method for obtaining the liquid Moringa peregrina protein hydrolysate, the protein hydrolysate is purified by distillation, microfiltration, ultrafiltration and/or nanofiltration in order to concentrate it into compounds of interest over the organic material that is also extracted, in particular over the other derivatives that are also extracted. These purification steps can be used to concentrate the pool of compounds of interest over the other extracted compounds that have been cited.

In accordance with another preferred embodiment of the method in accordance with the invention, nanofiltration is carried out in a manner such as to separate 3 bands or fractions of the protein hydrolysate, comprising a band P1 for which the molecular weight is less than 10000 Da, a band P2 for which the molecular weight is comprised between 10000 and 17 000 Da and a band P3 for which the molecular weight is approximately 23 000 Da.

It is also advantageous to separate the three bands by nanofiltration in order to obtain, by the method for obtaining a protein hydrolysate:
a band P1 with nanofiltration carried out with a cutoff threshold comprised between 1500 Da and 5000 Da, preferably with a cutoff threshold comprised between 3000 Da and 4500 Da.
a band P2 with nanofiltration carried out with a cutoff threshold comprised between 10000 Da and 17000 Da.
a band P3 with nanofiltration carried out with a cutoff threshold comprised between 17000 Da and 25000 Da.

In another embodiment of the extraction method in accordance with the invention, the liquid protein hydrolysate obtained is dried in a manner such as to obtain a dry hydrolysate of Moringa peregrina seed cake containing a quantity of more than 10%, preferably more than 20%, more preferably more than 30% and possibly up to approximately 40% (Weight/Weight) dry matter of peptides, oligopeptides, glycopeptides and amino acids or their volatile nitrile derivatives, more preferably 50% by weight of dry matter.

In accordance with one embodiment of the invention, the liquid protein hydrolysate from Moringa peregrina seed cake obtained is dried for example by atomization, lyophilization or zeodration in a manner such as to obtain a solid hydrolysate of Moringa peregrina seeds, the water being evaporated off. Drying may be carded out in the presence of an organic support such as maltodextrin, cyclodextrin or inulin, or in the presence of an inorganic support such as phyllosilicate, magnesium silicate or carbonate and its salts.

The invention also concerns the protein hydrolysate from Moringa peregrina seed cake obtained by the extraction method in accordance with the invention.

In a second aspect, the invention concerns a protein hydrolysate from seed cake that has not been shelled and has been harvested from ripe Moringa peregrina fruit, comprising a major fraction P1 of amino acid derivatives, amino acids, peptides and glycopeptides for which the molecular weight is comprised between 1500 Da and 5000 Da, a fraction P2 of approximately 20% for which the molecular weight is comprised between 10000 and 17000 Da and a fraction P3 of approximately 20% for which the molecular weight is approximately 23000 Da, in that it is obtained by chemical proteolysis at a pH of more than 13 for a period of approximately 2 hours at a temperature comprised between 16° C. and 25° C. and in that it is liquid and has a density of more than 1 and preferably about 1.1.

In accordance with one embodiment, the liquid protein hydrolysate obtained is dried in a manner such as to obtain a dry hydrolysate from Moringa peregrina seed cake containing a quantity of more than 20%, more preferably more than 30% and possibly up to approximately 40% (Weight/Weight) dry matter of peptides, oligopeptides, glycopeptides and amino acids or their volatile nitrile derivatives, more preferably 50% by weight of dry matter.

In accordance with a preferred embodiment, the protein hydrolysate also comprises between 0.3% and 3% of volatile compounds, wherein 50% of these compounds, i.e. between 0.15% and 1.5% of the extract in accordance with the invention, is constituted by light nitrile compounds, principally with isobutyronitrile and methylbutanenitrile; wherein 5% to 10% of these compounds, i.e. between 0.015% and 0.3% of the extract in accordance with the invention, is constituted by isothiocyanate derivatives, principally with isopropyl isothiocyanate and isobutyl isothiocyanate; wherein 1% to 5% of these compounds, i.e. between 0.003% and 0.15%, is constituted by essential oil, principally with eucalyptol, menthol and benzaldehyde.

In accordance with yet another embodiment, the protein hydrolysate comprises a dry matter content comprised between 10% and 15%, preferably about 12.5%, comprising between 1% and 6% of nitrogen-containing compounds, in particular volatile nitrile derivatives in a proportion of 0.5% to 1.5%, preferably about 0.8%, as well as 20 mg/liter of polyphenols.

In the context of the present invention, the portion of the plant that is selected is the Moringa peregrina seed. It is known that Moringa peregrina seeds are used in order to extract theft oil, which is useful for domestic consumption or for various traditional medicinal treatments. The cake obtained after the seed has been defatted is a waste product that is currently used to feed animals in particular.

In accordance with yet another embodiment, the protein hydrolysate comprises the major fraction P1 for which the molecular weight is comprised between 1500 Da and 5000 Da.

In accordance with yet another embodiment, the protein hydrolysate comprises the fraction P2 of approximately 20% for which the molecular weight is comprised between 10000 Da and 17000 Da.

In accordance with yet another embodiment, the protein hydrolysate comprises the fraction P3 of approximately 20% for which the molecular weight is approximately 23000 Da.

In accordance with yet another embodiment, the protein hydrolysate comprises the fraction P1 for which the molecular weight is comprised between 1500 Da and 5000 Da and the fraction P2 for which the molecular weight is comprised between 10000 and 17000 Da.

In a third aspect, the invention concerns a protein hydrolysate from Moringa peregrina seed cake for its application as a drug.

In a fourth aspect, the invention concerns a pharmaceutical or dermatological composition comprising, as the active agent, an effective quantity of a protein hydrolysate from Moringa peregrina seed cake in accordance with the invention and a physiologically acceptable excipient.

The compositions in accordance with the invention may be in any of the galenical forms that are in normal use, depending upon whether the composition is to be ingested, injected or applied to the skin or to the mucous membranes.

In accordance with a first variation, the various compositions are suitable for ingestion; the composition may be in the form of capsules, syrups, granules or tablets. It does not have to comprise any excipient and could be constituted in its entirety by the plant extract comprising the protein hydrolysate in the dry form.

In accordance with a second variation, the various compositions are suitable for injection; the composition may be in the form of an aqueous or oily lotion, or in the form of a serum.

In accordance with a third variation, the various compositions are more particularly suitable for local administration, enabling a pharmacological effect to be obtained at the exact site of the disorder via the skin or the mucous membranes.

The principal local modes used for administration of the active agent in accordance with the invention are the cutaneous and percutaneous modes, the nasal and respiratory modes, the ocular mode, the auricular mode and the vaginal mode. Other modes may be envisaged, in particular the buccal mode, for the administration of the compositions via the mucous membranes as well as the subcutaneous mode by micro-injections.

The compositions in accordance with the invention for pharmaceutical use may be in any of the galenical forms that are normally used for application via the local route. They may be administered to the mucous membranes, in particular by nasal or respiratory, ocular, buccal, vaginal and auricular administration.

The compositions in accordance with the invention for dermatological use may be in any of the galenical forms that are normally used in the dermatological field for cutaneous application. They may be administered transdermally or applied topically to the skin.

The compositions incorporating a protein hydrolysate in accordance with the invention may comprise ingredients that are routinely used in this type of formulation for local or cutaneous administration.

In accordance with a preferred embodiment, the various compositions are suitable for topical administration and include creams, oil-in-water and water-in-oil emulsions, milks, ointments, lotions, oils, balms, aqueous or hydroalcoholic or glycolic solutions, serums, powders, patches, sprays or any other product for external application such as, for example, medical devices or aerosol products that also contain a propellant under pressure.

In accordance with another preferred embodiment, the various compositions are suitable for subcutaneous injection and transdermal administration; the composition may be in the form of an aqueous lotion, emulsion or in the form of a serum. In transdermal or patch systems, the release of the active principle or principles is controlled by a permeable membrane that is generally adhesive and in direct contact with the skin.

The compositions in accordance with the invention that are more particularly intended for local administration contain an acceptable pharmaceutical or dermatological medium, i.e. compatible with the skin and the mucous membranes, and encompass all of the appropriate galenical forms. These compositions may in particular be in the form of creams, oil-in-water emulsions or water-in-oil or multiple emulsions, serums, solutions, suspensions, gels, milks, lotions, sticks, aerosols, sprays or any other product for external application such as, for example, medical devices or aerosol products also containing a pressurized propellant, or in fact any form of powder that is suitable for application to the skin and the mucous membranes. These compositions comprise the excipients that are necessary to their formulations, such as solvents, emollients, thickeners, diluents, surfactants, antioxidants, bioactive agents, colorants, preservatives, fragrances.

The compositions in accordance with the invention therefore comprise any additive that is in routine use in the envisaged field of application, as well as the adjuvants that are necessary to their formulations, such as solvents, thickeners, diluents, antioxidants, colorants, sunscreens, self-tanning agents, pigments, fillers, preservatives, fragrances, odor absorbers, dermatological or pharmaceutical active agents, essential oils, vitamins, essential fatty acids, surfactants, film-forming polymers, etc.

In all cases, the person skilled in the art will take care to ensure that these adjuvants as well as their proportions are selected so that they do not have a deleterious effect on the desired advantageous properties of the compositions in accordance with the invention.

In the compositions of the invention, the protein hydrolysate in accordance with the invention is used in a quantity ranging from 0.0001% to 40% by weight with respect to the total weight of the composition.

In a preferred embodiment, the protein hydrolysate in accordance with the invention is used in a quantity ranging from 0.001% to 10% by weight with respect to the total weight of the composition, more preferably in a quantity ranging from 0.01% to 5% by weight with respect to the total weight of the composition.

Finally, in a fifth aspect, the invention concerns a pharmaceutical or dermatological composition for its use as a drug for the treatment of:
fibrotic diseases and the treatment of inflammation comprising, as the active agent, an effective quantity of the fraction P1 or P2 of the protein hydrolysate, preferably the fraction P1.
cancer, comprising, as the active agent, an effective quantity of the fraction P3 of the protein hydrolysate.
infectious diseases of the bacterial or viral type, and in particular in order to inhibit spike-COV2 proteins, comprising, as the active agent, an effective quantity of the protein hydrolysate as a whole.
genetic drift and pathologies associated with skin pigmentation, comprising, as the active agent, an effective quantity of fractions P1 and P2 of the protein hydrolysate. The term “genetic drift” in particular means environmental stress. The term “pathologies associated with pigmentation” means carcinomas and lentigo, for example.

For the treatment of fibrotic diseases, the term fibrosis means pulmonary fibrosis, renal fibrosis, hepatic fibrosis, cutaneous fibrosis, ocular fibrosis, cardiac fibrosis and other various fibrotic states and for the treatment of other furin-mediated pathological states, in particular but not limited to hypertension, cancer, infectious diseases including viral and bacterial diseases, genetic disorders (for example, cystic fibrosis (CF)), and neurodegenerative disorders.

While the invention has been described with respect to several particular embodiments, it is clear that it is not in any way limited to these and it includes any technical equivalents of the means described as well as their combinations if they fall within the scope of the invention.

The use of the verb “compose of”, “comprise” or “include” and its conjugated forms does not exclude the presence of elements or steps other than those that have been defined in a claim.

### EXAMPLES

### Example 1: Preparation of a Plant Protein Hydrolysate in Accordance With the Invention Starting From Moringa peregrina Cake

Seeds of Moringa peregrina (Forssk.) Fiori were dried in order to obtain an internal moisture content of less than 8% and preferably about 6%, then pressed with an endless screw mechanical press in a manner such as to separate the oil from the remainder of the seed in order to obtain virgin oil on the one hand and cake on the other hand. The cake was then isolated in the form of extrudates precut into 1 to 2 cm pieces, on which the extraction was carried out.

The starting materials used were as follows.

TABLE 1

| Material | % | Actual quantity pressed |
|---|---|---|
| Peregrina cake | 8.4% | 33.6 |
| precut into 1 to 2 cm pieces | | |
| Mains water | 80.68% | 322.7 |
| Sodium hydroxide | 3.36% | 13.5 |
| Citric acid monohydrate F6000 | 7.14% | 28.7 |
| Sodium benzoate | 0.42% | 1.7 |
| | 100 | 400.2 |

### Protocol:

a) a concentrated 1 molar solution of a strong alkali such as sodium hydroxide, potassium hydroxide or calcium hydroxide in particular, or an aqueous mixture with a 1 molar concentration of strong alkalis, but preferably sodium hydroxide, was prepared; this strong alkaline solution had a pH of between 13 and 14,
b) 9.1% (weight/weight) of peregrina cake precut into 1 cm pieces was weighed into approximately 90.9% (weight/weight) of the alkaline solution,
c) the cake obtained in step b) was ground,
d) The ground cake obtained in step c) was dispersed in an aqueous phase,
e) the aqueous dispersion obtained in step d) underwent chemical proteolysis for a period of 2 hours and at a temperature of 22° C.,
f) the proteolysis was neutralized in order to stabilize the protein hydrolysate obtained,
g) the protein hydrolysate was recovered by solid/liquid separation by passage through a 1 μm filter,
h) the protein hydrolysate was purified by ultrafiltration,

A translucent yellow filtrate containing approximately 12.48% of dry matter was obtained. The liquid extract obtained is known below as the “peregrina protein hydrolysate in accordance with the invention” or the “peregrina protein hydrolysate” or the “extract in accordance with the invention”.

This peregrina protein hydrolysate in accordance with the invention had a density of more than 1 and preferably about 1.1, comprising a dry matter content comprised between 10% and 15%, preferably about 12.5%, comprising between 1% and 6% of nitrogen-containing compounds, in particular volatile nitrile derivatives, in a proportion of 0.5% to 1.5%, preferably about 0.8%, and 20 mg/liter of polyphenols. The composition of the dry peregrina protein hydrolysate in accordance with the invention is given below.

TABLE 2

| Rt | CAS No. | Compounds | % DM |
|---|---|---|---|
| 4.42. | 64-17-5 | Ethanol | 0.809 |
| 5.58 | 67-64-1 | Acetone | 0.444 |
| 6.10 | 75-15-0 | Carbon disulfide | 0.327 |
| 7.83 | 78-93-3 | 2-butanone | 0.165 |
| 8.57 | 141-78-6 | Ethyl acetate | 0.047 |
| 8.99 | 78-82-0 | Isobutyronitrile | 20.720 |
| 10.39 | 78-82-0 | Isobutyronitrile | 0.127 |
| 12.06 | 547-63-7 | Methyl isobutyrate | 0.201 |
| 14.10 | 18936-17-9 | 2-methylbutanenitrile | 2.194 |
| 14.56 | 625-28-5 | 3-methylbutanenitrile | 27.495 |
| 18.77 | 66-25-1 | Hexanal | 0.186 |
| 21.09 | 2253-73-8 | Isopropyl isothiocyanate | 4.590 |
| 23.65 | 628-73-9 | Hexanenitrile | 0.198 |
| 24.43 | 110-43-0 | 2-Heptanone | 0.093 |
| 27.18 | 4426-79-3 | 2-butyl isothiocyanate | 1.058 |
| 27.59 | 80-56-8 | Alpha-pinene | 0.048 |
| 28.45 | 591-82-2 | Isobutyl isothiocyanate | 2.299 |
| 28.99 | 100-52-7 | Benzaldehyde | 1.753 |
| 29.77 | 108-95-2 | Phenol | 0.413 |
| 30.80 | 13475-82-6 | 2,2,4,6,6-pentamethylheplane | 0.318 |
| 32.80 | 99-87-6 | Para-cymene | 0.232 |
| 33.10 | 138-86-3 | Limonene | 0.133 |
| 33.34 | 470-82-6 | Eucalyptol | 0.918 |
| 36.45 | 1195-32-0 | Para-cymenene | 0.035 |
| 36.88 | 124-19-6 | Nonanal | 0.080 |
| 40.26 | 65-85-0 | Benzoic acid | 19.150 |
| 41.07 | 1490-04-6 | Menthol | 0.918 |
| 56.29 | 96-76-4 | 2,4-Di-tert-butylphenol | 0.086 |
| | | Total | 85.035 |

The peregrina protein hydrolysate contains relatively high levels of isopropyl isothiocyanate and isobutyl isothiocyanate, confirming previous publications on the species (KJAER, A. et al. 1979, Isothiocyanates in Myrosinase-treated seed extracts of Moringa peregrina, Phytochemistry, 18, p. 1485-1487; AFSHARYPUOR, S. et al., 2010, Volatile Constituents of the Seed Kernel and Leaf of Moringa peregrina (Forssk.) Fiori, Agricolt. Cultivated in Chabahar (Iran), Iranian Journal of Pharmaceutical Sciences 6 (2): p. 141-144; DEHSHAHRI, S. et al., 2012, Determination of volatile glucosinate degradation products in seed coat, stem and in vitro cultures of Moringa peregrina (Forssk.) Fiori, ScienceOpen, Research in Pharmaceutical Sciences 7 (1): p. 51-56). Isothiocyanates are compounds produced by various plants belonging to the Brassicales order, in particular in the Brassicaceae, Capparaceae, Caricaceae and Moringaceae families, as a defense system against attacks by pathogens. In the genus Moringa, they have been identified in particular in M. oleifera Lam. and M. stenopetala (Baker f.) Cufold. (ABD RANI, N. Z. et al., 2018, Moringa genus: A review of Phytochemistry and Pharmacology, Frontiers in Pharmacology, vol. 9, art. 108, p. 3-8). The isothiocyanates derive from the hydrolysis of glucosinolates by the enzyme myrosinase when the tissues of the plant become damaged. It has been reported that isothiocyanates have a variety of biological effects, such as an antifungal activity (TRONCOSO-ROJAS, R. et al., 2007, Natural compounds to control fungal diseases in fruits & vegetables, in TRONCOSO-ROJAS, R., TIZNADO-HERNÁNDEZ, M. E., GONZÁLEZ-LEÓN, A. (ed) Recent advances in alternative postharvest technologies to control fungal diseases in fruits & vegetables. Transworld Research Network, Kerala, India, p. 127-156; TRONCOSO-ROJAS, R. et al., 2005, Analysis of the isothiocyanates present in cabbage leaves extract and their potential application to control Alternaria rot in bell peppers, Food Research International 38, p. 701-708), antimicrobial, anti-cancer and anti-inflammatory effects (PARK, E. J. et al., 2011, Inhibition of lipopolysaccharide induced cyclooxygenase-2 expression and inducible nitric oxide synthase by 4-[(2′-O-acetyl-α-L-rhamnosyloxy)benzyl]isothiocyanate from M. oleifera, Nutrition and Cancer 63 (6), p. 971-982; RAJAN T. S. et al., 2016, Anticancer activity of glucomoringin isothiocyanate in human malignant astrocytoma cells, Fitoterapa 110, p. 1-7; P_{ADLA,} E. P. et al. 2012, Antimicrobial isothiocyanates from the seeds of Moringa oleifera Lam., Zeitschrift für Naturforschung C, 67, p. 557-564; WATERMAN, C. et al., 2014, Stable, water extractable isothiocyanates from Moringa oleifera leaves attenuate inflammation in vitro. Phytochemistry 103, p. 114-122). Furfural is present in a highly standard concentration that is found in this type of seeds and in many dried fruits. Carbon disulfide, isobutyronitrile, methyl isobutyrate, methyl butanenitrile and hexanenitrile are volatile compounds obtained from amino acids. They are markers for the degradation of proteins. These degradation compounds represent approximately 50% of the volatile compounds of the peregrina protein hydrolysate. This result indicates that the hydrolysate is principally constituted by proteins. Only very slight traces of fats or free sugars have been detected in the peregrina protein hydrolysate; the hydrolysate is principally constituted by proteins and glycoproteins. In the dry material, the citrate buffer (which is an aside compound) represents approximately 50% of the remainder and encompasses the glycosylated compounds (osides) resulting from the degradation of the proteins, oligopeptides and amino acids by proteolysis. Finally, the presence of approximately 21 mg/liter of polyphenols (0.002%) obtained from seeds of Moringa peregrina should be noted. Benzoic acid should not be considered in the characterization because it is a stabilizing agent added to the extract.

The dry extract described above was obtained by a gravimetric method based on the mass present in the liquid extract before and after evaporation.

### Example 2: Effect of the peregrina Protein Hydrolysate in Accordance With the Invention as an Inhibitor for Furin Convertase (Known as Furin)

The aim of this study was to evaluate the inhibiting activity of the protein hydrolysate from Maringa peregrina seed cake obtained in accordance with Example 1.

Protocol: The study was carried out using a recombinant human furin, which catalyzes cleavage of a specific fluorescent reference substrate.

100 nM decanoyl-Arg-Val-Lys-Arg-CMK was used as a reference inhibitor for the activity of furin.

The furin was pre-incubated for 10 minutes at ambient temperature in the absence (control) or in the presence of a reference product, or of increasing concentrations of the test compound:

“Peregrina protein hydrolysate”; 0.02; 0.2 and 2% (v/v).

At the end of the pre-incubation step, a furin substrate was added and the experimental conditions were incubated once again at ambient temperature away from light for 5 minutes. All of the experiments were carried out in triplicate.

### Preparation of Compounds

The test peregrina protein hydrolysate was dissolved directly in the assay buffer then diluted in order to obtain the test concentration described above.

### Evaluation Protocol

Cleavage of the fluorescent furin substrate was monitored for 5 minutes after addition of the substrate by reading the fluorescence at 485 nm/535 nm.

### Statistics

The results are expressed in RFU (relative fluorescent units)+/−S.D. (standard deviation).

The statistical significance of the difference observed between the groups “Control” and “Reference product” was evaluated by a Student t test (p<0.001).

The statistical significance of the difference observed between the groups “Control” or “Reference” and “Test compound” was evaluated by a one-way ANOVA, followed by a Holm-Sidak test (p<0.05).

The reference inhibitor for furin, termed decanoyl-Arg-Val-Lys-Arg-CMK, tested at 100 nM, significantly inhibited the activity of the furin by 97.9% (p<0.001).

This result was expected and validated the study.

The results for the activity of the furin enzyme compared with the base activity obtained are given below.

TABLE 3

| | | |
|---|---|---|
| “Peregrina protein | 124.9% | (p < 0.001) |
| hydrolysate” at 0.02% (v/v) | | |
| “Peregrina protein | 73.2% | (p < 0.001) |
| hydrolysate” at 0.2% (v/v) | | |
| “Peregrina protein | 1.2% | (p < 0.001) |

hydrolysate” at 2.0% (v/v)

Conclusion: At a concentration of 2%, the peregrina protein hydrolysate in accordance with the invention is capable of inhibiting 98.8% of the activity of furin convertase. Beyond a concentration of 0.2%, the peregrina protein hydrolysate in accordance with the invention is capable of inhibiting the activity of furin by 26.8%.

### Example 3: Effect of the peregrina Protein Hydrolysate in Accordance With the Invention in Inhibiting Histone Deacetylases (HDACs) and Sirtuin I Enzymes

The aim of this study is to demonstrate the inhibiting activity of the peregrina protein hydrolysate in accordance with the invention on the HDAC and sirtuin I enzymes, enzymes that are involved in the control of genetic drift by the regulation of the condensation/decondensation of chromatin, which provides or prohibits access to genes carried by DNA. A buffered solution of HDACs and sirtuin I reacts with a substrate over 20 minutes at 37° C. and transforms it in order to form a compound that colors in the presence of a developer after incubation at 37° C. for 10 minutes. The maximum activity for deacetylation of sirtuins can then be evaluated by measuring the absorbance at 405 nm. The peregrina protein hydrolysate in accordance with the invention or the reference product “Trichostatin A (STA) inhibitor 1 μM” were brought into contact with the solution of sirtuins at the same time as the substrate for the enzyme for 20 minutes at 37° C.; the substrate transformed by the enzyme was stained by the addition of a developer. The deacetylating activity of the HDACs and sirtuin I in the presence of the active ingredient was then evaluated by measuring the absorbance at 405 nm. The modulation of this activity was expressed as a percentage inhibition or activation of the maximum activity of the HDACs and sirtuin I in the absence of active ingredient, i.e. solely in the presence of the substrate for the HDACs & sirtuin I enzymes.

Protocol: A solution of sirtuin enzymes was incubated in its substrate for 20 min in the absence (control) or presence of the reference product, or in increasing concentrations of the products to be tested. The peregrina protein hydrolysate in accordance with the invention was tested at the following concentrations: 2%; 1%; 0.1% (V/V). At the end of the incubation period, the activity of the sirtuin enzymes with and without the test product or reference product was revealed by staining using a developer solution (10 min at 37° C.) and evaluated by measuring the absorbance of the reaction media at 405 nm. For each test concentration, the modulation of the deacetylating activity of the histone deacetylase and sirtuin I enzymes by the test product was calculated using the following formula.

Percentage modulation of the activity of sirtuin enzymes=100×[(OD₄₀₅ produced in test or reference)−(0D405 HDACs & sirtuin I alone)]/OD₄₀₅ sirtuins alone. [Math. 1]

If the result is negative, the percentage is expressed as the inhibition of the enzymatic reaction; if the result is positive, the percentage is expressed as the activation of the enzymatic reaction. The results for the inhibition of the histone deacetylase enzymes (HDACs) are given below.

TABLE 4

| | Percentage | Inhibition versus Control (%) |
|---|---|---|
| Peregrina protein | 2% | 15 |
| hydrolysate in | 1% | ns |
| accordance with | 0.10% | −18 |
| the invention | | |

Conclusion: At 2%, the peregrina protein hydrolysate in accordance with the invention exhibits significant inhibition of HDACs; this inhibition reveals the capacity for promoting self-protection of cells of the skin against genetic drift. Thus, the extract appears to be useful against one of the most routine genetic drifts on the surface of the skin, namely fibrosis, which is manifested by the appearance of a “lump” of flesh (fibrotic protuberance). The extract could advantageously interfere in fibrosis phenomena on the surface of the skin.

### Example 4: Effect of the peregrina Protein Hydrolysate in Accordance With the Invention in Order to Inhibit the Action of Endothelin 1 (ET-1)

Endothelin is a hormone peptide derived from endothelial cells that is capable of acting on various cells and tissues via its receptors. As an example, endothelin is known to cause an increase in the intraceliular concentration of calcium in smooth muscle vascular cells and other cells (OHBA T. et al., WO 2012/081370).

It has been reported in recent years that endothelin type 1 (ET-1) is a bioactive factor that contracts the smooth vascular and non-vascular muscle cells by direct and indirect actions. It is considered that an increase in the action of endothelin provides continuous vasoconstriction to blood vessels in peripheral sites, in the kidneys and in the brain, and could be at the origin of a variety of diseases such as hypertension, myocardial infarction, cerebrovascular accidents, acute renal insufficiency, Raynaud syndrome, atherosclerosis, asthma and prostate cancer (MIYAGAWA K. & EMOTO N. et al., 2014, Current state of endothelin receptor antagonism in hypertension and pulmonary hypertension, Therapeutic Advances in Cardiovascular Diseases, vol. 8 (5) 202-216; SCHINZARI F. et al., 2018, Increased Endothelin-1-Mediated Vasoconstrictor Tone in Human Obesity: Effects of Gut Hormones, Physiological Research 67 suppl.1: S69-S81). Three types of peptides from the endothelin family with similar structures are present in animals, including human beings (KADONO, S. et al., 2001, The Role of the Epidermal Endothelin Cascade in the Hyperpigmentation Mechanism of Lentigo Senilis, Journal of Investigative Dermatology 116 4, p. 571-577; ANONYMOUS, 2006, American Society for Biochemistry and Molecular Biology, New Cosmetics Handbook, p. 527-529). All of these peptides have a vasoconstrictive effect and vasopressive actions.

Recently, the role of endothelin in a variety of cells other than smooth vascular muscle cells has been elucidated. As an example, scientific publications have reported that the generation of ET-1 and other factors increases in keratinocytes when the skin is exposed to UV radiation and have suggested that ET-1 might be associated with melanogenesis in melanocytes exposed to UV radiation. As a consequence, the suppression of the expression of endothelin is considered to be useful, not only for the prevention and/or treatment of the aforementioned diseases, but also in order to prevent or improve pigmentation of the (IMOKAWA, G. et al., 1995, Endothelin-1 as a New Melanogen: Coordinated Expression of its Gene and the Tyrosinase Gene in UVB-Exposed Human Epidermis, Journal of Investigative Dermatology 1051, p. 32-37; IMOKAWA, G. et al., 1992, Endothelins Secreted from Keratinocytes are Intrinsic Mitogens for Human Melanocytes, Journal of Biological Chemistry, 267, p. 24675-24680; GILCHREST, B. et al., 1996, Mechanisms of Ultraviolet Light-Induced Pigmentation, Photochemistry and Photobioliogy 63, p. 1-10).

The aim is to assay endothelin type 1 in human micro-vascular endothelial cells after exposure to the peregrina protein hydrolysate in accordance with the invention for 24 h.

Protocol: The human micro-vascular endothelial cells were provided by PELOBiotech and cultured in 96-well plates in accordance with the supplier's production procedures. This meant allowing the extracts to act at different concentrations on endothelial cells at 80% confluence for 24 hours, then quantifying endothelin I in the cell supernatants with the aid of the ELISA PicoKine (EDN1) kit. A prior viability test was carried out in order to define the non-toxic doses to be used when assaying the endothelin 1. The negative control was formed by cells in culture medium without treatment. The positive control in the viability test was 0.5% SDS. All of the conditions were prepared in culture media and the cells were then incubated at 36.5° C./5% CO₂ for 24 hours.
a) Application of test solutions to endothelial cells:
The test products were brought into contact with the sub-confluence endothelial cells in 96-well plates. The test was carried out on 3 wells for each concentration. The plates were incubated for 24 hours±1 hour at 36.5° C./5% CO₂.
b) Viability test:
Cell viability was evaluated using the MTT method on the cells after incubation with the products. After 24 hours of incubation, the supernatants were recovered and stored at −20° C. for the assays. The wells were then rinsed once with 200 μL of PBS. 50 μL of 0.5 mg/mL MTT solution was added to each well: incubation was carried out for 3 hours at 36.5° C./5% CO₂. 100 μL of isopropanol was added to each well. After homogenization, the absorbance was recorded at 550 nm. For each condition, the ratio of the mean of the optical densities of the cells to the mean of the optical densities of the negative controls would determine the viability ratio.
c) Endothelin 1 assay:
The assay was carried out with the aid of an ELISA kit. The results for the inhibition of the action of endothelin are given below.

TABLE 5

| | | | | Endothelin 1 |
|---|---|---|---|---|
| | Extract | Cell growth versus | Endothelin 1 versus | versus control |
| | concentration | control (%) | control (%) | (pg/mL) |
| Protein | 2% | +8.46 | −34.90 | −47.09 |
| hydrolysate | | | | |
| of the | 1% | 1.54 | −13.03 | −17.58 |
| invention | 0.10% | −1.15 | −2.44 | −16.79 |

Conclusion: The viability test carried out at the end of treatment did not exhibit any toxic effects for the concentrations that were tested.
The endothelin 1 assay was carried out on the cell supernatants at non-toxic concentrations. The quantity of endothelin 1 for each condition was assayed with the aid of the ELISA kit.
For the base level control cells, the values were of the order of 134.94 μg/mL. For the cells treated with different concentrations of hydrolysate, the base level was reduced by 47.09 μg/mL (with 2% of the extract in accordance with the invention) and reduced by 17.58 μg/mL (with 1% of the extract in accordance with the invention). This demonstrates the very significant inhibitions from 1% of the extract in accordance with the invention with approximately 13% of inhibition of the production of endothelin type 1 and up to 34.90% inhibition with 2% of the extract in accordance with the invention.
   The results for endothelin and its specific dose-dependent inhibition demonstrate that the protein hydrolysate in accordance with the invention has an anti-angiogenesis and anti-fibrotic effect because of its capacity to significantly reduce endothelin.

A complementary study for the evaluation of P1, P2, P3 and of the complex P1+P2 described in Example 12 is described below for the production of endothelins: Cell test model on normal human endothelial cells.

As described in Example 12, 3 bands or protein fractions were isolated from the hydrolysate in accordance with the invention, characterized by theft mass P1 (at a molecular weight of less than 10000 Da), P2 (at a molecular weight comprised between 10000 and 17000 Da) and finally P3 (at a molecular weight of approximately 23000 Da). These fractions are hereinafter also termed “extracts”.

TABLE 6

| | | Extract P1 (%, v/v) | | |
|---|---|---|---|---|
| | Reference | 0.1 | 0.3 | 1 |
| Endothelin-I (pg/ | 33.8 | 29.0 | 44.0 | 38.3 |
| μg of proteins) | 36.7 | 34.2 | 41.5 | 31.7 |
| | 39.7 | 30.1 | 38.1 | 33.9 |
| Mean | 36.7 | 31.1* | 41.2* | 34.6* |
| Standard | 3.0 | 2.7 | 2.9 | 3.3 |
| deviation | | | | |
| % of reference | 100.0 | 84.6 | 112.1 | 94.2 |

*Statistical significance (p > 0.05)

At all concentrations, the standard deviations were high and the fraction P1 of the protein hydrolysate did not exhibit any significant modulation on endothelin type 1.

TABLE 7

| | | Extract P2 (%, v/v) | | |
|---|---|---|---|---|
| | Reference | 0.1 | 0.3 | 1 |
| Endothelin-I (pg/ | 33.8 | 34.8 | 24.7 | 34.6 |
| μg of proteins) | 36.7 | 35.7 | 28.9 | 39.3 |
| | 39.7 | 34.0 | 29.2 | 40.8 |
| Mean | 36.7 | 34.9* | 27.6** | 38.3* |
| Standard | 3.0 | 0.9 | 2.5 | 3.2 |
| deviation | | | | |
| % of reference | 100.0 | 94.9 | 75.2 | 104.1 |

*Statistical significance (p > 0.05)
**Statistical significance (p < 0.05)

At concentrations of 0.1% and 1%, the standard deviations were high, and the fraction P2 of the protein hydrolysate did not exhibit any significant modulation on endothelin type 1 at these concentrations. At the concentration of 0.3%, P2 inhibits the production of endothelin type 1 by 24.8%.

TABLE 8

| | | Extracts P1 + P2 (50/50) (%, v/v) | | |
|---|---|---|---|---|
| | Reference | 0.1 | 0.3 | 1 |
| Endothelin-I (pg/μg | 33.8 | 39.4 | 42.0 | 34.5 |
| of proteins) | 36.7 | 29.7 | 34.2 | 34.7 |
| | 39.7 | 42.2 | 38.3 | 37.9 |
| Mean | 36.7 | 37.1* | 38.1* | 35.7* |
| Standard | 3.0 | 6.6 | 3.9 | 1.9 |
| deviation | | | | |
| % of reference | 100.0 | 100.9 | 103.8 | 97.2 |

*Statistical significance (p > 0.05)

At all concentrations, the standard deviations were high, and the combination of fractions P1+P2 did not exhibit any significant modulation on endothelin type 1.

TABLE 9

Extract P3 (%, v/v)

| | Reference | 0.1 | 0.3 | 1 |
|---|---|---|---|---|
| Endothelin-I (pg/μg | 33.8 | 36.2 | 19.5 | 18.8 |
| of proteins) | 36.7 | 38.9 | 14.8 | 20.4 |
| | 39.7 | 31.5 | 18.2 | 17.6 |
| Mean | 36.7 | 35.5* | 17.5*** | 18.9*** |
| Standard deviation | 3.0 | 3.8 | 2.4 | 1.4 |
| % of reference | 100.0 | 96.7 | 47.7 | 51.5 |

*Statistical significance (p > 0.05)
***Statistical significance (p < 0.001)

At concentrations of 0.3% and 1%, the fraction P3 of the protein hydrolysate very significantly inhibits approximately 50% of the production of endothelin type 1.

Conclusion: The compounds termed “Extract P1” and “Extract P2” did not have a significant action on the modulation of the endothelin type 1, and only the “Extract P3” significantly reduced the endothelin-1 liberated into the culture medium by the normal human endothelial cells in a monolayer culture with a score of 52.3% inhibition with 0.3% of the extract in accordance with the invention. No synergistic effects were observed when P1 and P2 were combined.

The extract P3 specifically exhibited an anti-cancer effect (BAGNATO A. et al., 2011, Role of the endothelin axis and its antagonists in the treatment of cancer, British Journal of Pharmacology, 163:220-233).

### Example 5: Effect of the peregrina Protein Hydrolysate in Accordance With the Invention for the Protection of Stem Cells

Adult tissues, including the cutaneous epidermis, the gastro-intestinal epithelium and the hematopoietic system, have a very high level of cell renewal. The physiological process of maintaining tissue homeostasis is attributed to maintaining a constant number of cells in organ renewal. Embryonic stem cells (ESC) are essential to maintaining and regenerating cutaneous tissues.

The epidermis develops from the ectodermis of the embryo surface. It starts as a single layer of non-specific progenitor cells covering the embryo after neurulation and develops into the basal epidermal layer. The basal epidermal layer is enriched in ESCs (Epidermal Stem Cells). In fact, the cells of this layer give rise to all of the epidermal structures, including the stratified epidermis (also known as the interfollicular epidermis) and the epidermal appendices, such as the hair follicles, the sebaceous glands and the sweat glands. The subjacent dermis principally derives from the mesodermis under the ectodermis. The mesodermis is the principal source of mesenchymal stem cells, which give rise to the collagen-producing fibroblasts, the subjacent adipocytes and the immune cells of the skin.

Stem cells are undifferentiated cells that are known as pluripotent cells with a young genotype that is capable of self-renewal and of differentiating to produce an organ or a tissue such as the skin. At this stage, they are identified as “multipotent cells”. Given that 50% of the descendants of the stem cell population remains undifferentiated, the stem cells contribute to preserving homeostasis and ensuring renewal of damaged or senescent differentiated cells. However, these epidermal stem cells are frequently affected by the environment. Oxidative stress such as pollution or ultraviolet radiation damages their DNA, according to YEJIN GE et al. (10 Mar. 2020, The aging skin microenvironment dictates stem cell behavior, PNAS, Vol. 117, p. 5339-5350). This damage alters their capacity for self-renewal and differentiation, leading to a reduction in the pool of stem cells, and finally to aging of the skin.

The aim of the study is to evaluate the effect of the peregrina protein hydrolysate in accordance with the invention on the protection of the epidermal stem cells against UVB irradiation.

Protocol: Human keratinocyte cells were obtained from a 62 year old donor. In order to carry out the experiments, the keratinocytes were cultured in a monolayer until they reached 80% confluence.

The cell culture was then enriched in epidermal stem cells by following the method described by GOODELL, M. et al. (1996, Hoescht 33342 HSC staining and stem cell purification protocol, Journal of Experimental Medicine 183, p. 1797-806).

Reference product: 1 μM quercetin was used as a reference product in this study. The quercetin was purchased from Sigma Aldrich.

The cells were pre-incubated for 24 hours in the absence (“Control”) or in the presence of the reference product or an increasing concentration of the compound to be tested. At the end of the pre-incubation period, the cells were irradiated with UVB (30 mJ/cm²) then incubated for 8 days at 37° C. in the absence (reference) or in the presence of the reference product or by increasing the concentration of the compound to be tested.

“Peregrina protein hydrolysate”: 0.01; 0.05 and 0.15% (v/v).

### Preparation of Test Compound:

The test compound “peregrina protein hydrolysate” was diluted directly in the incubation medium in order to obtain the various concentrations described above.

At the end of the incubation period, the cell viability was measured using Alamar blue, a non-cytotoxic viability indicator based on the reduction of rezazurin by mitochondria. Each experimental condition was carried out in triplicate (n=3).

The results are presented below as a percentage viability with respect to the “Control without UVB” experimental condition (mean+/−S.D). The level of significance between “Control without UVB” and “Control with UVB” was evaluated with the aid of a Student test (p<0.05).

TABLE 10

| | | Cell growth | I Protection of stem |
|---|---|---|---|
| | Concentration | versus control | cells versus control |
| | of hydrolysate | (%) | (%) |
| Peregrina | 0.15% | ca. −9.2 | NS |
| protein | 0.05% | −3.6 | +30.50 |
| hydrolysate | 0.01% | ca. −6 | +15.50 |

Conclusion: The peregrina protein hydrolysate can significantly protect the stem cells of the human skin subjected to cellular stress (UV). Stem cells are cells with preserved and young DNA material. They are at the origin of tissue regeneration, and a return to a young and healthy state. The protection of stem cells is correlated with the capacity to preserve DNA material. The peregrina protein hydrolysate in accordance with the invention maintains the integrity of the stem cells; it is therefore involved in DNA conversion.

### Example 6: Effect of the peregrina Protein Hydrolysate in Accordance With the Invention on Preserving DNA

The dynamics of the length of telomeres is very important for the regulation of the replicative lifetime of cells, in particular in species that are long-lived. Shortening of telomeres and the activity of telomerase are important factors in aging and in tumorigenesis (SHAY, J. W. & WRIGHT, W. E., 2005, Senescence and Immortalization: Role of Telomeres and Telomerase, Carcinogenesis 26 (5), p. 867-874). Telomeres are complex nucleotide sequences that cover the ends of chromosomes for degradation, unwanted fusion-recombination, inappropriate activation of the response to damage to DNA. They also play an essential role in cell division and the stability of chromosomes. More and more evidence exists that the stability of telomeres and their mean length can be affected by stress, in particular environmental stress, or diseases under environmental influence. (VALDES, A. L. et al., July 2005, Obesity, cigarette smoking and telomere length in women, Research Letters. 366 (9486), p. 662-664; PHILLIPS A. C. et al., 2013, Do symptoms of depression predict telomere length? Evidence from the West of Scotland Twenty-07 Study, Psychosomatic Medicine, 75 (3), p. 288-296; SHIN, D. et al. May 2019, Effects of inflammation and depression on telomere length in young adults in the United States, Journal of Clinical Med 2019, 8 (5), p. 711; SALIQUES, S. et al., October 2010, Telomer length and cardiovascular disease, Archives of Cardiovascular Diseases, 103 (8-9), p. 454-459). Thus, extremely short telomeres have been associated with neurodegenerative diseases, cardiovascular disease (CVD) and the risk of cancer.

Telomerase is a ribonucleoprotein that catalyzes the addition of telomeric repetitions to the ends of the telomeres. Telomeres are long sections of repeated sequences that cap the ends of the chromosomes and are known to stabilize the chromosome. In human beings, the telomeres are generally 7 to 10 kb in length and include several repetitions of the sequence -TTAGGG-.

Telomerase is not expressed in the majority of adult cells and the length of the telomeres reduces with successive replication cycles. After a certain number of replication cycles, the progressive shortening of the telomeres causes the cells to enter a phase of telomeric crises, which in turn leads to cell senescence. Certain diseases are associated with a rapid loss of telomeres, causing premature cell senescence. It has been shown that the expression of the gene coding for the human telomerase protein in human cells (BLASCO M., 2007, Telomere Length, Stem Cells and Aging, Nature Chemical Biology, 3 (10), p. 640-649) produces a phenotype with a constant quality, probably by reversing the route to natural senescence of the cells. In addition, it has been demonstrated in the study cited above that the expression of the gene for telomerase in aging cells with short telomeres produces an increase in the length of the telomeres and restores a phenotype that is generally associated with younger cells.

The aim of this study is to evaluate the effect of the compound termed “peregrina protein hydrolysate” on telomere shortening in a model composed of normal human fibroblasts in a monolayer culture. It is well known that the telomere corresponds to a biological clock. The length of the telomeres reduces progressively with cell divisions, in the end resulting in a cell that is incapable of replication. The measurement of the length of the telomeres was carded out using quantitative PCR and comparison with the length of the telomeres among cells at passes 2 and 5.

Protocol: Human fibroblast cells were obtained from a 44 year old donor. In order to carry out the experiments, the cells were used in passes 2 and 5. The fibroblasts were cultured for 3 consecutive passes in the absence (control) or in the presence of an increasing test concentration of the peregrina protein hydrolysate: 0.01%; 0.1% and 0.5% (v/v).

Preparation of test compound: The “peregrina protein hydrolysate” test compound was diluted directly in the incubation medium in order to obtain the various concentrations described above.

At the end of the incubation, the cells were trypsinized. The DNA was extracted from the cells with the aid of a dedicated DNA extraction kit. The DNA was quantified by nanodrop.

The telomere length was measured by quantitative PCR (q-PCR). For each sample, the variation in the telomere length was measured by relative quantification using the SCR (single copy reference) gene as a reference gene. For each sample, a q-PCR was carried out using a set of telomere primers which recognize and amplify the telomere sequences and a second q-PCR was carried out using the set of SCR primers which recognize and amplify a 100 bp region on the human chromosome 17 and act as a reference for standardization of the data.

The results are expressed in relative units corresponding to the length of the telomeres with respect to the cells in pass 2 (mean±S.D.). The level of significance versus “Control” in passes 2 and 5 was evaluated with the aid of a Student test (*: p<0.05). The level of significance between “control” and “test compound” was evaluated independently for each product by a single factor analysis of variance (one-way ANOVA) followed by a Holm-Sidak test (*: p<0.05).

TABLE 11

| | | Cell growth | Length of telomeres |
|---|---|---|---|
| | Concentration | versus control (%) | versus control (%) |
| Peregrina | 0.5% | +1.9 | +16.60 |
| protein | 0.1% | ca. 0 | +15.10 |
| hydrolysate | 0.05% | -0.6 | +8.90 |

Results: Under our experimental conditions, the peregrina protein hydrolysate tested at 0.05%, 0.1% and 0.5% (v/v), significantly reduced shortening of normal human fibroblast telomeres.

The shortening of the telomeres exhibited an inhibition (compared with the control) at 0.05% (v/v) of +8.9% (p<0.05); at 0.1% (v/v) of +15.1% (p<0.01) and at 0.5% (v/v) of +16.6% (p<0.01). Conclusion: in a context of normal multiplication or division of human cells, the peregrina protein hydrolysate in accordance with the invention demonstrates a capacity to significantly increase the length of telomeres. Telomeres are plugs involved in the protection of DNA material; increasing the length of the telomeres is correlated with the capacity to preserve DNA material. The peregrina protein hydrolysate can increase the length of the telomeres. This hydrolysate is therefore involved in the preservation of human genetic material (DNA).

Complementary study of the evaluation of extracts P1, P2, P3 and of the complex P1+P2 described in Example 12 on their capacity to protect DNA by increasing the length of telomeres after several cell divisions.

TABLE 12

| | | Pass 5 | | | |
|---|---|---|---|---|---|
| | Pass 2 | | Extract P1 (%; v/v) | | |
| | Reference | Reference | 0.01% | 0.3% | 1% |
| Variation in | 1.08 | 0.56 | 0.57 | 0.65 | 0.74 |
| telomere length | 0.86 | 0.58 | 0.61 | 0.64 | 0.77 |
| (change in size | 1.07 | 0.54 | 0.62 | 0.71 | 0.70 |
| with respect to | | | | | |
| first pass) | | | | | |
| Mean | 1.00 | 0.56 | 0.60* | 0.67** | 0.74*** |
| Standard | 0.12 | 0.02 | 0.03 | 0.04 | 0.04 |
| deviation | | | | | |
| Telomere Length | 100.0 | 55.8 | 59.8 | 66.6 | 73.3 |
| (as % of | | | | | |
| reference) | | | | | |
| % elongation | | 0 | 9.1 | 24.3 | 39.6 |
| of telomeres | | | | | |
| compared with | | | | | |
| reference | | | | | |

*Statistical significance (p > 0.05)
**Statistical significance (p < 0.01)
***Statistical significance (p < 0.001)

After 3 cell divisions from the concentration of 0.3%, the extract P1 in accordance with the invention increased the size of the telomeres on a normal human cell culture model (fibroblasts) by 24.3%. At the concentration of 1%, under the same conditions, the extract P1 increased the telomere size by 39.6%.

TABLE 13

| | | Pass 5 | | | |
|---|---|---|---|---|---|
| | Pass 2 | | Extract P2 (%; v/v) | | |
| | Reference | Reference | 0.01% | 0.3% | 1% |
| Variation in | 1.08 | 0.56 | 0.53 | 0.61 | 0.66 |
| telomere length | 0.86 | 0.58 | 0.70 | 0.71 | 0.68 |
| (change in size | 1.07 | 0.54 | 0.74 | 0.68 | 0.92 |
| with respect to | | | | | |
| first pass) | | | | | |
| Mean | 1.00 | 0.56 | 0.66* | 0.67* | 0.75* |
| Standard | 0.12 | 0.02 | 0.11 | 0.05 | 0.14 |
| deviation | | | | | |
| Telomere Length | 100.0 | 55.8 | 65.3 | 66.3 | 75.0 |
| (as % of | | | | | |
| reference) | | | | | |
| % elongation | | 0 | 21.4 | 23.7 | 43.5 |
| of telomeres | | | | | |
| compared with | | | | | |
| reference | | | | | |

*Statistical significance (p > 0.05)

After 3 cell divisions on a normal human cell culture model (fibroblasts), the extract P2 in accordance with the invention had a statistically unconfirmed tendency for elongation of the telomeres for each test concentration.

TABLE 14

| | | Pass 5 | | | |
|---|---|---|---|---|---|
| | Pass 2 | | Extract P3 (%; v/v) | | |
| | Reference | Reference | 0.01% | 0.3% | 1% |
| Variation in | 1.08 | 0.56 | 0.51 | 0.46 | 0.50 |
| telomere length | | | | | |
| (change in size | 0.86 | 0.58 | 0.52 | 0.54 | 0.55 |
| with respect to | | | | | |
| first pass) | 1.07 | 0.54 | 0.64 | 0.61 | 0.61 |
| Mean | 1.00 | 0.56 | 0.56* | 0.54* | 0.55* |
| Standard deviation | 0.12 | 0.02 | 0.07 | 0.07 | 0.06 |
| Telomere Length | 100.0 | 55.8 | 55.4 | 53.6 | 55.2 |
| (as % of reference) | | | | | |
| % elongation | | 0 | −0.8 | −5.0 | −1.3 |
| of telomeres | | | | | |
| compared with | | | | | |
| reference | | | | | |

*Statistical significance (p > 0.05)

After 3 cell divisions on a normal human cell culture model (fibroblasts), the extract P3 in accordance with the invention did not have any capacity to promote elongation of the telomeres for each test concentration.

TABLE 15

| | | Pass 5 | | | |
|---|---|---|---|---|---|
| | Pass 2 | | Extract P1 + P2 (%; v/v) | | |
| | Reference | Reference | 0.01% | 0.3% | 1% |
| Variation in | 1.08 | 0.56 | 0.61 | 0.72 | 0.76 |
| telomere length | 0.86 | 0.58 | 0.84 | 0.67 | 0.71 |
| (change in size | 1.07 | 0.54 | 0.63 | 0.74 | 0.86 |
| with respect to | | | | | |
| first pass) | | | | | |
| Mean | 1.00 | 0.56** | 0.70* | 0.71* | 0.78** |
| Standard deviation | 0.12 | 0.02 | 0.13 | 0.04 | 0.08 |
| Telomere Length | 100.0 | 55.8 | 69.3 | 70.5 | 77.5 |
| (as % of reference) | | | | | |
| [symbol] ongation | | 0 | 30.5 | 33.3 | 49.1 |
| of telomeres | | | | | |
| compared with | | | | | |
| reference | | | | | |

*Statistical significance (p > 0.05)
**Statistical significance (p < 0.01) [symbol]
indicates data missing or illegible when filed

After 3 cell divisions at the concentration of 1% (i.e. 0.5% for each extract), the mixture of extracts P1 and P2 (50/50 by volume) in accordance with the invention increased the size of the telomeres on a normal human cell culture model (fibroblasts) by 49.1%. This score was not reached by the individual extracts, and so a synergistic effect has been demonstrated by mixing the extract P1 and P2.

Conclusion: The compounds termed “Extract P1” and “Extract P1+P2” significantly reduce the shortening of telomeres, occurring after 3 consecutive cell passes. A synergistic effect is confirmed with a combination of P1 and P2.

### Example 7: Effect of the peregrina Protein Hydrolysate in Accordance With the Invention to Stimulate the ZAG Protein

Zinc alpha-2-glycoprotein (ZAG) is a plasma glycoprotein that takes its name from its electrophoretic mobility and its capacity to be precipitated by Zn salts. ZAG forms part of the superfamily of immunoglobulin genes and has a three-dimensional structure which is highly homologous to CMH class I and II molecules. ZAG has been detected immunohistochemically in normal secretory epithelial cells of the breast, prostate and liver, in the saliva glands, bronchial, gastro-intestinal and sweat glands and in normal stratified epitheliums including the epidermis. The mRNA of ZAG remains uniformly distributed in the various types of cells (FREIJE, J. P. et al., 1991, Human Zn-α2-Glycoprotein cDNA Cloning and Expression Analysis in Benign and Malignant Breast Tissues, FEBS Letters 290 (1-2), p. 247-249). Because it is produced by the secretory epithelium, ZAG is present in the majority of bodily secretions and respectively constitutes 2.5% of the proteins in saliva and 30% of seminal fluids. It has been reported that the levels of ZAG in plasma and serum vary with age, with the reported values ranging from 0.9 to 3.5 mg/dl (fetus) to 7.8 to 12.1 mg/dl (young adults) (JIRKA, M. et al., 1974, The Zn-alpha 2-Glycoprotein Level in Human Serum During Ontogenesis. Clinica Chimica Acta 56, p. 31-33; JIRKA, M. et al., 1978, Human Serum Zn-α2-Glycoprotein in Amniotic Fluid, Clinica Chimica Acta 85, p. 107-110). ZAG accumulates in breast cyst fluid up to a plasma concentration of 30 to 50 times (BUNDRED et al., 1987, An Immunohistochemical Study of the Tissue Distribution of the Breast Cyst Fluid Protein, Zinc Alpha2-Glycoprotein, Histopathology 11, p. 603-610; DIEZ-ITZA, I. et al., 1993, Zn-α2-Glycoprotein Levels in Breast Cancer Cytosols and Correlation with Clinical, Histological, and Biochemical Parameters, European Journal of Cancer 29A, p. 1256-1260) and is overexpressed in 40% to 50% of breast carcinomas. It has recently been demonstrated (SUSAN, M. el al., Zinc-alpha2-glycoprotein Expression as a Predictor of Metastatic Prostate Cancer Following Radical Prostatectomy, 2006, Journal of the National Cancer Institute, Volume 98 (19), p. 1420-1424) that ZAG is produced in quantity by the majority of prostate carcinomas, which causes an elevation in serum levels of ZAG in patients with prostate cancer in the basocellular carcinomas. The aim of this study is to evaluate the capacity of the peregrina protein hydrolysate to stimulate ZAG.

Protocol: Normal human keratinocytes were isolated from foreskin and cultured in 24 and 96 well plates in accordance with internal procedures.

This involved leaving the samples to act on the keratinocytes at 80% confluence for 48 hours at defined concentrations, then quantifying the ZAGs in the cell supernatants with the aid of the ELISA kit.

A prior viability test was carried out in order to define non-toxic doses to be used when assaying the ZAGs. The negative control was produced with the aid of cultured cells without treatment. The positive control for the viability test was 0.5% SDS.

All of the conditions were prepared in the culture media, and the cells were then incubated at 36.5° C./5% CO₂ for 24 hours for the viability test and 48 hours for the assay of the ZAGs.
Application of test solutions to the keratinocytes:
   The test products were brought into contact with the keratinocytes in sub-confluence in the 24 and 96 well plates.
   For each concentration, the test was carried out on 3 wells.
   The plates were incubated for 24 hours and 48 hours at 36.5° C./5% CO₂.
Viability test:
   The cell viability was evaluated using the MTT method on the cells after incubation with the products.
   After 24 and 48 hours of incubation, the wells provided were rinsed once with 2004 of PBS.
   50 μL of MTT solution at 0.5 mg/mL was added to each well: incubation was for 3 hours at 36.5° C./5% CO₂.
   100 μL of isopropanol was added to each well.
   After homogenization, the absorbance was recorded at 550 nm.
   For each condition, the ratio of the mean of the optical densities of the cells to the mean of the optical densities of the negative controls would determine the viability ratio.
Assay of ZAG proteins:
   After 48 hours of incubation, all of the supernatants were recovered and stored at −20° C. for the assays.
   The assay was carried out with the aid of an ELISA kit. The results are given below.

TABLE 16

| | | Cell growth | | |
|---|---|---|---|---|
| | | versus | ZAG | ZAG versus |
| | Concentration | control (%) | versus | control (ng/ml) |
| Peregrina | 2% | −17.88 | +337.80 | 0.390 |
| protein | 1% | −12.54 | +195.73 | 0.157 |
| hydrolysate | 0.10% | −3.88 | +151.83 | 0.085 |

Conclusion: The peregrina protein hydrolysate can significantly increase the production of ZAG, with good dose dependency and a low toxicity on human cells. For this reason, it has an anti-fibrotic effect and an anti-inflammatory effect based on its capacity to significantly increase ZAG.

A complementary study of the stimulation of ZAG starting from the protein bands identified in Example 12 is described below for normal human keratinocytes.

TABLE 17

| | | Extract P1 (%, v/v) | | |
|---|---|---|---|---|
| | Reference | 0.2 | 1 | 5 |
| ZAG (pg/μg | 2.8 | 4.1 | 4.3 | 2.3 |
| of proteins) | 3.0 | 6.0 | 3.9 | 1.1 |
| | 2.0 | 6.1 | 3.4 | 2.4 |
| Mean | 2.6 | 5.4** | 3.9* | 1.9* |
| Standard | 0.5 | 1.1 | 0.5 | 0.7 |
| deviation | | | | |
| % of | 100.0 | 207.1 | 147.3 | 73.5 |
| reference | | | | |

*Statistical significance (p > 0.05)
**Statistical significance (p < 0.01)

From 0.2%, the extract P1 significantly increased the production of ZAG by more than 107%.

TABLE 18

| | | Extract P2 (%, v/v) | | |
|---|---|---|---|---|
| | Reference | 0.2 | 1 | 5 |
| ZAG (pg/μg of | 2.8 | 6.2 | 4.3 | 2.6 |
| proteins) | 3.0 | 4.2 | 4.4 | 2.7 |
| | 2.0 | 3.7 | 4.5 | 2.3 |
| Mean | 2.6 | 4.7** | 4.4** | 2.5* |
| Standard | 0.5 | 1.4 | 0.1 | 0.2 |
| deviation | | | | |
| % of reference | 100.0 | 180.1 | 167.5 | 95.6 |

*Statistical significance (p > 0.05)
**Statistical significance (p < 0.05)

From 0.2%, the extract P2 significantly increased the production of ZAG by more than 80%.

TABLE 19

| | | Extract P3 (%, v/v) | | |
|---|---|---|---|---|
| | Reference | 0.2 | 1 | 5 |
| ZAG (pg/μg of | 2.8 | 2.1 | 2.3 | 2.3 |
| proteins) | 3.0 | 1.9 | 2.7 | 1.8 |
| | 2.0 | 2.2 | 2.1 | 3.3 |
| Mean | 2.6 | 2.1* | 2.4* | 2.5* |
| Standard | 0.5 | 0.2 | 0.3 | 0.8 |
| deviation | | | | |
| % of reference | 100.0 | 78.5 | 90.0 | 95.2 |

*Statistical significance (p > 0.05)

The extract P3 did not have the capacity to significantly influence the production of ZAG in this study model.

TABLE 20

| | | Extract P1 + P2 (50/50) (%, v/v) | | |
|---|---|---|---|---|
| | Reference | 0.2 | 1 | 5 |
| ZAG (pg/μg of | 2.8 | 3.9 | 4.2 | 1.7 |
| proteins) | 3.0 | 3.7 | 3.5 | 2.6 |
| | 2.0 | 3.5 | 2.8 | 2.6 |
| Mean | 2.6 | 3.7* | 3.5* | 2.3* |
| Standard deviation | 0.5 | 0.2 | 0.7 | 0.5 |
| % of reference | 100.0 | 140.6 | 132.9 | 87.8 |

*Statistical significance (p > 0.05)

The combination of extracts P1 and P2 exhibited a tendency to activation of the production of ZAG, but this tendency was not significant from a statistical viewpoint. Thus, there was no synergistic effect in the combination of the extracts P1 and P2.

The compounds termed “Extract P1” and “Extract P2” significantly increased the ZAG liberated into the culture medium by normal human keratinocytes in a monolayer culture, but no synergistic effects were observed when combining P1 and P2.

Conclusion: In the hydrolyzed extract in accordance with the invention, the extract P1 is the extract that performs best for increasing ZAG. For this reason, it has an anti-fibrotic effect and an anti-inflammatory effect from a dose of 0.2%.

### Example 8: Effect of the peregrina Protein Hydrolysate in Accordance With the Invention on The Modulation of DKK1 and DKK3 Assays

The implications of the interactions between melanocytes and fibroblasts in the regulation of melanogenesis is well known and has been studied in depth. Although these interactions are not yet fully understood, they are at the origin of the “bleaching” of palmoplantar zones and are now used in dermatology for the development of depigmentation products. Yamaguchi et al (YAMAGUCHI Y. et al., 2004, Mesenchymal-Epithelial Interactions in the Skin: Increased Expression of Dickkopf by Palmoplantar Fibroblasts Inhibits Melanocyte Growth and Differentiation, Journal of Cell Biology 165 (2), p. 275-285) have demonstrated that a soluble messenger produced by the fibroblasts of the palmoplantar regions are capable of modifying the melanocyte differentiation program for these regions, leading to a reduction in the production of melanin. This messenger was identified by the team as a protein known as Dickopf-1 (DKK-1).

The signaling pathways used by DKK-1 to produce these results have now been identified. Because of its antagonist action on the Wnt receptor, DKK-1 is in fact capable of short-circuiting the intracellular signaling pathways activated by β-catenin, generally responsible for the regulation of the genes involved in melanogenesis. Yamaguchi et al (cf. supra) have also demonstrated that DKK-3, a molecule like DKK-1 but with no effect on the Wnt receptor, could play a regulatory role on the DKK-1 effect. In fact, the larger the quantity of DKK-3 in the vicinity of this Wnt receptor, the weaker are the interactions between DKK-1 and this receptor on melanogenesis. The work by Yamaguchi et al (cf. supra) also suggest that the identification of agents having an influence on the DKK1/DKK3 ratio in cultures of normal human dermal fibroblasts of non-palmoplantar origin could control the production of melanin starting from normal non palmoplantar human melanocytes.

The aim of this study is to evaluate the effect of the peregrina protein hydrolysate on the synthesis and release of DKK-1 in a model compound of normal human fibroblasts in a monolayer culture.

Protocol: Human fibroblast cells were obtained from a 68 year old donor. In order to carry out the experiments, the fibroblasts were cultured in a monolayer to confluence. 100 nM dexamethasone was used as a reference inducer for the synthesis and release of DKK-1. The disks of skin were incubated for 48 hours in the absence (control) or in the presence of the reference product or of the test product: “peregrina protein hydrolysate”: 0.01%; 0.1% and 0.5% (v/v).

At the end of the incubation, the incubation media were removed in order to carry out the DKK-1 liberation method.

The “peregrina protein hydrolysate” test compound was diluted directly in the incubation medium in order to obtain the various concentrations described above.

At the end of the incubation period of 48 hours, the DKK-1 liberated into the incubation medium was quantified with the aid of a sensitive and specific ELISA kit.

At the end of the incubation period, the proteins contained in the cell lysates were quantified with the aid of a spectrocolorimetric method (Bradford method).

The results are expressed in ng of DKK-1 per mg of proteins (mean±S.D.).

The level of significance between the “control” and the “reference product” was evaluated with the aid of a Student test (p<0.05).

The level of significance between the “Control” and the “test product” was evaluated by a single factor analysis of variance (one-way ANOVA) followed by a Holm-Sidak test (p<0.05).

Under our experimental conditions, the reference product denoted “Dexamethasone”, tested at 100 nM, significantly increased the DKK-1 liberated by 181.8% (p<0.01) compared with the “Control”. The results on the modulation of the DKK1 assay are given below.

TABLE 21

| | | Cell growth versus | DKK1 versus |
|---|---|---|---|
| | Concentration | control (%) | control (%) |
| Peregrina | 0.5% | +1.9 | +131.50 |
| protein | 0.1% | ca. 0 | +32.30 |
| hydrolysate | 0.05% | −0.6 | +26.10 |

The study shows that the hydrolysate in accordance with the invention significantly increased the levels of DKK1 at a dose of 0.05% with a 26.1% increase with respect to the base level and at a concentration of 0.5% of the extract in accordance with the invention, an increase of 131.5% of the base level was observed.

The aim of this study was to evaluate the effect of the peregrina protein hydrolysate on the synthesis and the liberation of DKK-3 in a model compound of normal human fibroblasts in a rnonolayer culture. The human fibroblast cells were obtained from a 68 year old donor. In order to carry out the experiments, the fibroblasts were cultured in a monolayer to confluence. The human fibroblast cells were obtained from a 68 year old donor. In order to carry out the experiments, the fibroblasts were cultured in a monolayer to confluence.

At the end of the incubation period of 48 hours, the DKK-3 liberated into the incubation media was quantified with the aid of a sensitive and specific ELISA kit.

At the end of the incubation period, the proteins contained in the cell lysates were quantified with the aid of a spectrocolorimetric method (Bradford method).

The results are expressed in ng of DKK-3 per mg of proteins (mean±S.D.). The level of significance between the “Control” and the “reference product” was evaluated with the aid of a Student test (*: p<0.05).

The level of significance between the “Control” and the peregrina protein hydrolysate was evaluated by a single factor analysis of variance (one-way ANOVA) followed by a Holm-Sidak test (*: p<0.05). The results on the modulation of the DKK3 assay are given below.

TABLE 22

| | | Cell growth versus | DKK3 versus |
|---|---|---|---|
| | Concentration | control (%) | control (%) |
| Peregrina | 0.5% | 101.9 | −20.7 |
| protein | 0.1% | ca. 100.0 | −8 |
| hydrolysate | 0.05% | 99.4 | −3.9 |

Conclusion: The peregrina protein hydrolysate in accordance with the invention at 0.5% exhibited a substantial inhibition of the order of 21% for DKK3 compared with the base level. The peregrina protein hydrolysate in accordance with the invention has a large capacity to manage the genes involved in cell differentiation because of the palmoplantar inhibition principle (beta-catenin signaling pathway), by its capacity to significantly increase DKK1 and significantly reduce DKK3, which increases the ratio DKK1/DKK3.

Complementary study of the evaluation of P1, P2, P3 and of the complex P1+P2 described in Example 10 on the production of DKK1: Cell test model on normal human fibroblasts.

TABLE 23

| | | Extract P1 (%; v/v) | | |
|---|---|---|---|---|
| | Reference | 0.01 | 0.3 | 1 |
| DKK-1 | 306.7 | 334.8 | 399.5 | 372.9 |
| (ng/mg of | 294.9 | 293.1 | 347.9 | 392.0 |
| proteins) | 298.6 | 367.6 | 392.4 | 385.1 |
| Mean | 300.1 | 331.9* | 379.9** | 383.3** |
| Standard | 6.1 | 37.3 | 28.0 | 9.7 |
| deviation | | | | |
| % of reference | 100.0 | 110.6 | 126.6 | 127.8 |

*Statistical significance (p>0.05)
**Statistical significance (p<0.01)

The extract P1 significantly increased the production of DKK1 by 26.6% from the dose of 0.3%, and by 27.8% at a dose of 1%.

TABLE 24

| | | Extract P2 (%; v/v) | | |
|---|---|---|---|---|
| | Reference | 0.01 | 0.3 | 1 |
| DKK-1 | 306.7 | 291.5 | 292.6 | 410.8 |
| (ng/mg of | 294.9 | 346.5 | 275.4 | 367.0 |
| proteins) | 298.6 | 277.4 | 354.9 | 344.4 |
| Mean | 300.1 | 305.2* | 307.6* | 374.1* |
| Standard | 6.1 | 36.5 | 41.8 | 33.8 |
| deviation | | | | |
| % of reference | 100.0 | 101.7 | 102.5 | 124.7 |

*Statistical significance (p>0.05)

The extract P2 did not significantly statistically increase the production of DKK1 in any of the experimental doses.

TABLE 25

| | | Extract P3 (%; v/v) | | |
|---|---|---|---|---|
| | Reference | 0.01 | 0.3 | 1 |
| DKK-1 | 306.7 | 359.2 | 387.0 | 224.7 |
| (ng/mg of | 294.9 | 371.8 | 263.7 | 283.0 |
| proteins) | 298.6 | 293.4 | 287.3 | 288.6 |
| Mean | 300.1 | 341.5* | 312.7* | 265.5* |
| Standard | 6.1 | 42.1 | 65.5 | 35.4 |
| deviation | | | | |
| % of reference | 100.0 | 113.8 | 104.2 | 88.5 |

*Statistical significance (p>0.05)

The extract P3 did not significantly statistically increase the production of DKK1 in any of the experimental doses.

TABLE 26

Extract P1+P2 50/50(%; v/v)

| | Reference | 0.01 | 0.3 | 1 |
|---|---|---|---|---|
| DKK-1 | 306.7 | 305.9 | 366.6 | 389.8 |
| (ng/mg of | 294.9 | 311.1 | 357.1 | 479.5 |
| proteins) | 298.6 | 346.8 | 308.1 | 384.2 |
| Mean | 300.1 | 321.3* | 343.9* | 417.8** |
| Standard | 6.1 | 22.2 | 31.4 | 53.5 |
| deviation | | | | |
| % of reference | 100.0 | 107.1 | 114.6 | 139.2 |

*Statistical significance (p>0.05)
**Statistical significance (p<0.01)

The combination of the extracts P1 and P2 at the dose of 1% (i.e. 0.5% for each of the extracts) increased the production of DKK1 significantly and by more than 39%. This score is more than that obtained with the extract P1 or P2 alone; this therefore means that there is a synergistic effect in the combination of the two extracts P1 and P2.

Conclusion: The compounds termed “Extract P1” and “Extract P1+P2” significantly increased the DKK-1 liberated into the culture medium by normal human fibroblasts in a monolayer culture. A synergistic effect was confirmed when P1 and P2 were mixed.

### Conclusion Drawn From the Above Activities of the Protein Hydrolysate in Accordance With the Invention

The most remarkable activity of the peregrina protein hydrolysate demonstrated in the in celluia tests is its epigenetic action with its capacity to very significantly slow down the process of telomere shortening following cell division, The hydrolysate is a cellular protector, more particularly of stem cells; which makes them a very good cell and tissue regenerator. These properties offer very high protection of DNA and its genetic material. The peregrina protein hydrolysate is also a powerful modulator of the production of ZAG and of endothelin type 1, with an anti-fibrotic and anti-inflammatory effect.

### Example 9: Evaluation of the Effect of the Protein Hydrolysate From Delipidized peregrina Cake on the Angiotensin Convertase 2 (ACE2)—Acellular Study of the Inhibitor Effect

Angiotensin convertase 2 is in particular involved in intra-cellular infection by COVID19 after the activation of spike proteins by other convertases and more particularly by furin, see PETER BRADDING et al. (ACE, TMPRSS2, and furin gene expression in the airways of people with asthma—implications of COVID-19, JOURNAL ALLERGY CLINICAL IMMUNOLOGY, July 2020, n° 146 (1), p. 206-211)

TABLE 27

| | | | | % | |
|---|---|---|---|---|---|
| | | | Mean- | enzymatic | % |
| Samples | | Mean | blank | activity | inhibition |
| | Blank | 89576.000 | 0.000 | 0.00 | 100% |
| | T+ | 101450.333 | 11874.333 | 100.00 | 0% |
| | T− | 93017.667 | 3441.667 | 28.98 | 71% |
| Hydrolyzed | 2.0% | 98839.000 | 10687.333 | 90.00 | 10% |
| peregrina | 1.0% | 101400.667 | 17466.333 | 147.09 | NS |
| extract | 0.10% | 104515.333 | 24256.333 | 204.28 | NS |

Conclusion: The hydrolyzed extract of peregrina cake is not significantly involved in the direct inhibition of angiotensin convertase 2 (ACE2). By considering table of Example 11 below, the amplitude of the action of this extract on another convertase, namely furin, was evaluated. The inhibiting action of the extract in accordance with the invention on furin has been clearly demonstrated and is established in Table 32 of Example 11. This specific inhibition demonstrated on a single convertase enzyme reinforces the importance of the protein hydrolysate in accordance with the invention as a specific inhibitor for furin convertase.

### Example 10: Evaluation of the Anti-Infectious Effect of the Hydrolyzed Extract in Inhibiting Furin in Cellulo by Preventing the Penetration of “Spike SARS-CoV-2 Pseudotyped Coronavirus”: in Cellulo Model on Human HEK Cells in the Presence of “SARS-CoV-2 Pseudotyped Lentivirus”

The present evaluation used two principal components. On the one hand, stable recombinant clonal HEK293 cells constitutively expressing full length human ACE2 (Genbank #NM_021804.3) with a surface expression of ACE2 confirmed by flow cytometry. On the other hand, the spike SARS-CoV-2 pseudotyped lentivirus was produced with spike SARS-CoV-2 (Genbank access number QHD43416.1) as envelope glycoproteins instead of the VSV-G that is conventionally used. These pseudovirions also contain the gene for luciole luciferase directed by a CMV promoter; as a consequence, cell entry mediated by the peak can be measured in a practical manner via the activity of the luciferase reporter. The spike SARS-CoV-2 pseudotyped lentivirus may be used to screen the applications in a biosafety level 2 facility.

The materials used were as follows:

TABLE 28

| Catalogue | Components |
|---|---|
| 79951 | ACE2-HEK recomb cell line |
| 60187-1 | Medium 1 for thawing |
| 79801 | Growth medium 1N |
| 79942 | spike (SARS-CoV-2) Pseudotyped Lentivirus (Luciferase |
| | Reporter) |
| 79943 | Bald Lentiviral Pseudovirion (Luciferase Reporter) |
| 60690-1 | ONE-Step luciferase assay system |
| AF933 | Human/Mouse/Rat/Hamster Antibody ACE-2 (R&D |
| | Systems) |

### Step 1: Plating ACE2-HEK Cells

The ACE2-HEK cells were thawed in the thawing medium 1, amplified in the 1N growth medium then harvested and placed in white, transparent flat bottom 96-well culture plates, in an amount of 10000 cells/well in 50 μL of thawing medium 1. The cells were incubated overnight at 37° C.

### Step 2: Pseudotyped Lentivirus Infection Test

The following day, visual monitoring of the homogeneity and integrity of the cell layer was validated with the aid of an inverted microscope and the following test ingredients were prepared in accordance with the following instructions:

TABLE 29

| | Name of ingredient/co- | | Concentrations in |
|---|---|---|---|
| | incubation mode | Solubility | “well” |
| Ingredient | Peregrina/ACE2-HEK | H₂O/culture | 3% (C1), 0.5% (C2), |
| 1 | | medium | 0.1% (C3) |

In a first step, the peregrina protein hydrolysate was diluted 11× to an intermediate concentration in the thawing medium 1 and subsequently, 5 pt was transferred to the test plate and co-incubation with the ACE2-HEK cells was carried out. After an incubation period of 30 min at 37° C., 5 μL of undiluted pseudotyped lentivirus (Bald or S1-spike) was added to the corresponding wells for analysis.

The mAb blocking ACE2 was used as a positive control in a final concentration of 0.5 μM in the wells for analysis.

### Step 3:

After an incubation period of 48 hours, a volume of 50 μL of the reagent luciferase was added to the wells for analysis and the luminescent signal was measured using the PolarStar Omega luminometer.

The results are given in FIG. 1 for the anti-infectious effect of the peregrina protein hydrolysate in accordance with the invention against the pseudovirions of SARS COV2.

Conclusion: The peregrina protein hydrolysate exhibits a convincing anti-infectious effect on the pseudovirions of SARS COV2 starting from a dose of 3% (Ci) on the study model A marked inhibition of 60% at the concentration C1 (3%) has been demonstrated.

Evaluation of bands P1, P2, P3 identified in Example 12 and of the complex P1+P2 on furin convertase: acellular test model.

The studies carried out on the identified bands did not demonstrate any inhibiting action on furin convertase in all of the concentrations tested with P1, P2, P3 and P1+P2.

Conclusion: The inhibiting activity of furin convertase is therefore not carded by one of these protein bands. This demonstrates that the inhibiting activity of furin convertase is due to the protein hydrolysate in its entirety obtained by the method described.

The protein hydrolysate in its entirety inhibits the SARS-CoV2 spike type proteins, in particular by inactivation of furin convertase. These inhibitions provide an anti-infectious and virostatic nature.

### Example 11: Comparative Tests for the Inhibition of Furin Convertase by Different Preparations Obtained From Morinqa peregrina Seeds

The aim of this study was to evaluate the inhibiting effect of peregrina oil obtained by first cold pressing of seeds with shells, then of the peregrina extract with ethanol (96%) constituted by approximately 1.1% of dry matter, this dry matter itself being constituted by approximately 55% by weight 2,5-diformylfuran, 2.5% furfural, 1.2% isopropyl myristate, 4.7% palmitic acid, 11.1% oleic acid and 25.8% triglycerides, and finally the peregrina protein hydrolysate in accordance with the invention, on the activity of furin.

Protocol: The peregrina extract in ethanol (96%) and the peregrina protein hydrolysate were stored at +4° C., away from light until use. The peregrina oil was stored at ambient temperature in a dark area.

The reference product: 100 nM decanoyl-Arg-Val-Lys-Arg-CMK was used as the reference inhibitor for the activity of furin.

The incubation protocol: The furin was pre-incubated for 10 minutes at ambient temperature in the absence (Control) or in the presence of the reference product or increasing concentrations of the test compounds:
peregrina oil at 0.3%, 1% and 3% (v/v).
peregrina extract in ethanol (96%) at 0.02%, 0.2% and 2% (v/v).
peregrina protein hydrolysate in accordance with the invention at 0.02%, 0.2% and 2% (v/v). At the end of the pre-incubation step, a furin substrate was added and the experimental conditions were incubated again at ambient temperature, away from light, for 5 minutes. All of the experiments were carried out in triplicate.

Preparation of compounds: The peregrina extract in 96% ethanol and the peregrina protein hydrolysate were dissolved directly in the assay buffer then diluted in order to reach the test concentration, as described above. The peregrina oil was dissolved to 3% in a 0.05% Tween20 solution in assay buffer. The solution was then diluted in order to obtain the concentration described above.

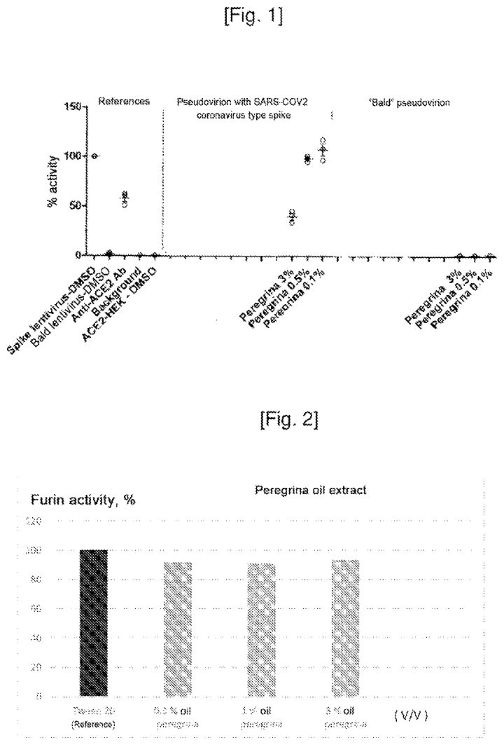

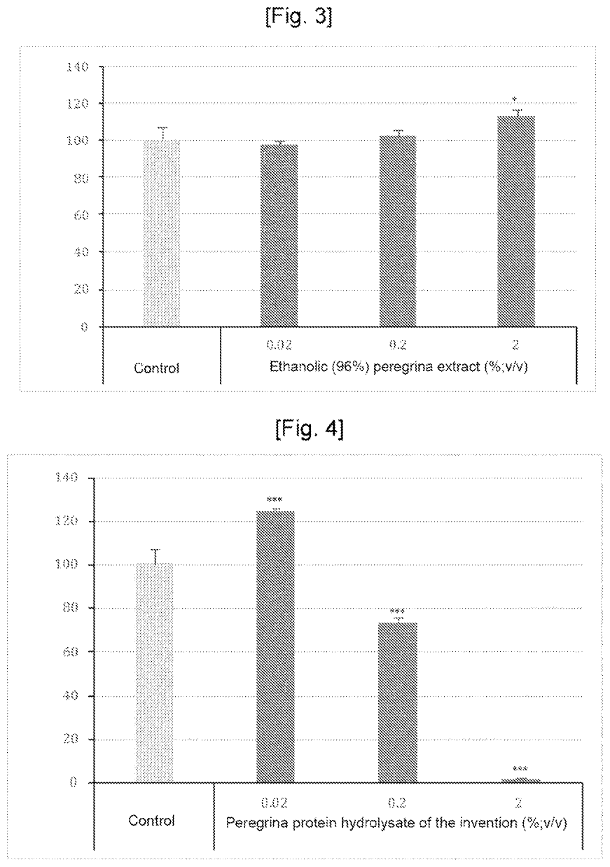

Evaluation protocol: Cleavage of the fluorescent furin substrate was monitored for 5 minutes after addition of the substrate by recording the fluorescence at 485 nm/535 nm. g. Statistics: The results are expressed in RFU (Relative Fluorescent Units)+/−S.D. (standard deviation). The statistical significance of the difference observed between the “Control” and “Reference product” groups was evaluated by a Student test (p<0.001). The statistical significance of the difference observed between the “Control” and “Test compound” groups was evaluated by a one-way ANOVA, followed by a Holm-Sidak test (p<0.05). The results for the peregrina oil are given in FIG. 2; those for the 96° ethanolic peregrina extract in FIG. 3 and finally for the peregrina protein hydrolysate in accordance with the invention are given in FIG. 4.

Results:

TABLE 30

| Concentration of peregrina oil | Activity of furin convertase |
|---|---|
| 0.3% (v:v) | 91.2%** |
| 1% (v:v) | 90.6%** |
| 3% (v:v) | 92.8%** |

TABLE 31

| Concentration of 96° | |
|---|---|
| ethanol peregrina extract | Activity of furin convertase |
| 0.02% (v:v) | 97.3%* |
| 0.2% (v:v) | 102.0%* |
| 2.0% (v:v) | 112.8%* |

TABLE 32

| Concentration of peregrina | |
|---|---|
| protein hydrolysate | Activity of furin convertase |
| 0.02% (v:v) | 124.9%*** |
| 0.2% (v:v) | 73.2%*** |
| 2.0% (v:v) | 1.2%*** |

*Statistical significance (p>0.05)
**: Statistical significance (p<0.01)
***: Statistical significance (p<0.001)

Conclusion: The present study enabled us to demonstrate that only the peregrina protein hydrolysate in accordance with the invention can significantly inhibit the activity of furin by 98.8% at a concentration of 2%.

### Example 12: Separative Electrophoresis Gel Chromatography of peregrina Protein Hydrolysate—Protein Bands

Gel electrophoresis on polyacrylamide containing sodium dodecyl sulfate is known as SDS-PAGE electrophoresis. It is a technique consisting of causing denatured proteins to migrate in a polyacrylamide gel by saturation of the negative charge via SOS under the influence of an electric field, enabling them to be separated thereby. It is a denaturing technique that dissociates non-covalent protein complexes by using a negatively charged ionic detergent (SOS). This detergent binds indiscriminately via hydrophobic bonds to two amino adds. This technique can therefore be used to analyze proteins and to separate them as a function of their molecular mass.

### Separative Chromatography by the Electrophoresis Method

### Deposits:

MW=size marker
well 1: hydrolyzed peregrina cake extract
well 2: supernatant of the hydrolyzed peregrina cake extract
wells 3: pellet of the hydrolyzed peregrina cake extract

The hydrolyzed extract in well 1 clearly exhibited all of the expected protein bands.

Well 2 (supernatant) exhibited the expected bands, in particular for the intermediate and highest molecular weights; well 2 appears to have concentrated the lowest molecular weights, in particular with the volatiles.

Well 3 (pellet) clearly exhibited the highest molecular weight bands (>75000 Da), then a band known as P3 of approximately 23000 Da, then a band known as P2 comprised between 10000 and 17000 Da and finally a band at less than 10000 Da known as P1, estimated to be between 4000 and 6000 Da.

### Rotary Evaporator Preparation

The volatiles were logically in the supernatant with the “lightest” compounds; 10 mL of supernatant was recovered and supplemented with 10 mL of 96° ethanol;
The mixture was extracted under vacuum at 45° C.; the volatile compounds were condensed in a vacuum flask.
The condensate, concentrated in volatiles, was passed through GC/FID after SPME micro-extraction.

### Gas Chromatography Study After a SPME Extraction Method With FID Detection

No volatile compounds were detected.

Conclusion: The isolation of the protein bands reveals that the volatile compounds are not bound to the proteins; the volatile compounds do not participate in the molecular cavalcade of the smallest protein bands.

Determination of three protein bands: The bands P1 (less than 10000 Da) and P2 (between 10000 and 17000 Da) and P3 (approximately 23000 Da) were prepared and passed on for liquid chromatographic analysis coupled with mass spectrometric analysis (LC MS/MS).

The results were obtained using two specialized software programs for protein recognition in plants (Mascot and Peaks):

The results of the identification did not enable these proteins to be identified precisely; we were in the presence of proteins which have not yet been described in the aforementioned databases.

### Example 13: Formulation for a Dermatological Anti-Fibrotic Product (Liquid Cleaner)

TABLE 31

| Ingredients | % |
|---|---|
| Water | 80.7000 |
| Sodium coco-sulfate | 5.0000 |
| Sodium cocoyl isethionate | 4.0000 |
| Bentonite | 3.7800 |
| Caprylic/Capric triglyceride | 2.0000 |
| Peregrina protein hydrolysate | 2.0000 |
| in accordance with the | |
| invention | |
| Gluconolactone | 0.7500 |
| Sodium benzoate | 0.5450 |
| Fragrance | 0.5000 |
| Xanthan gum | 0.2700 |
| Sodium stearoyl glutamate | 0.2250 |
| Citric acid | 0.2250 |
| Calcium gluconate | 0.0050 |

### Example 14: Formulation for a Dermatological Product Treating Topical Fibrosis (No-Rinse Care Product)

TABLE 32

| Ingredient | % |
|---|---|
| Water | 71.5000 |
| Caprylic/Capric triglyceride | 18.0000 |
| Bentonite | 4.2000 |
| Cetearyl alcohol | 1.5000 |
| Peregrina protein hydrolysate | 2.0000 |
| in accordance with invention | |
| Gluconolactone | 0.7500 |
| Sodium benzoate | 0.5450 |
| Xanthan gum | 0.5000 |
| Fragrance | 0.5000 |
| Sodium stearoyl glutamate | 0.2500 |
| Citric acid | 0.2500 |
| Calcium gluconate | 0.0050 |

### Example 15: Formulation for a Composition That can be Injected Subcutaneously

Formulation of a product for subcutaneous administration: dry extract in accordance with the invention (containing 60% of protein hydrolysate on an inulin support) packaged in a single dose flask under inert gas ready to be dissolved by a physiological medium.

### Example 16: Formulation for a Composition That can be Injected Subcutaneously

Formulation of an injectable liquid product: liquid extract in accordance with the invention in a 5% dose in a physiological medium packaged under sterilizing conditions in particular by vacuum filtration with a cutoff threshold of 0.45 μm.

### Example 17: Formulation in the Form of a Patch (Dressing or External Medical Device Adhered to the Skin, impregnated with active principle that is Slowly Released to Provide an Effect of Slow Diffusion of the Active Principle)

### Example 18: Formulation for a Drug in a Capsule

Antifibrotic drug in a 750 mg capsule containing 100 mg of piperine, 300 mg dry extract in accordance with the invention (containing 60% of protein hydrolysate on an inulin support), 100 mg boswellic acid, 250 mg of calcium carbonate).

### Example 19: Formulation for a Drug as a Tablet

Antifibrotic drug in 1 g tablet: 300 mg of dry extract in accordance with the invention (containing 60% of protein hydrolysate on an inulin support), 400 mg of calcium carbonate containing 200 IU of vitamin D, 150 mg of magnesium gluconate, 80 mg of inulin and 70 mg of magnesium stearate.

### Example 20: Formulation for a Drug as a Nasal Spray (Solution Containing the Active Principle in a Medical Device Which Propels the Active Principle by Spraying the Active Solution Into the Nasal Cavity)

### Example 21: Toxicological Tests on the Protein Hydrolysate in Accordance With the Invention

Preparation of the protein hydrolysate in accordance with Example 1: Unshelled seeds of Moringa peregrina (Forssk.) Fiori harvested when the fruit was ripe were dried in order to obtain an internal moisture content of less than 8% and preferably about 6%, then pressed with an endless screw mechanical press in a manner such as to separate the oil from the remainder of the seed in order to obtain the virgin oil on the one hand and a cake on the other hand. The cake was then isolated in the form of extrudates that had been cut into 1 to 2 cm pieces. By following the protocol described in Example 1, the liquid extract that was obtained was used undiluted in the following tests.

### 1. Determination of Mutagenic Activity on the Bacterial Strain Salmonella typhimurium (TA 100)—Reverse Bacterial Mutation Test

The test was carried out in 3 principal phases:
A preliminary experiment was carried out in order to evaluate the cytotoxicity of the element to be tested and to select the range of doses for the subsequent experiments,
A first genotoxicity experiment (Test 1), with and without metabolic activation, with direct incorporation of the test system and the test (or controls) onto minimum gel, over the range of doses defined in the preliminary study,
A second experiment (Test 2), with pre-incubation of the test system and the test (or controls), with and without metabolic activation, with dose levels defined by the person in charge of the study after analysis of the results from the first experiment. This second experiment was carried out in order to confirm or supplement the results from the first experiment, in particular when equivalent or negative results were obtained. Dilutions of the extract in accordance with Example 1 were prepared in water in order to carry out the cytotoxicity test

The cytotoxicity test was carried out on the Salmonella typhimurium TA100 strain at concentrations of 5000, 1600, 500, 160 and 50 μg/plate, with and without S9-Mix.

The reagents used for the preparation of the S9-Mix were prepared in accordance with the following instructions:

TABLE 33

| | Final concentration |
|---|---|
| MgCl₂ (0.4M) + KCl (1.65M) | 8 mM + 33 mM |
| Glucose 6 Phosphate (0.2M) | 5 mM |
| NADP (0.1M) | 4 mM |
| Phosphate buffer for S9-Mix | 0.1 M |
| (pH 7.4 - 0.2 M) | |
| S9 fraction | 10% |
| Water | Adjust for final concentration |

The bacteria were exposed to the test extract of the invention with and without the metabolic activation system. The metabolic system used was a post-mitochondrial fraction improved with cofactor (S9). This fraction 59, a microsomal fraction of Sprague Dawley rat liver homogenate treated with an enzymatic inducer, was prepared in accordance with MARON, D. M. et al., 1983, Revised Methods for the Salmonella Mutagenicity Test, Mutation Research/Environmental Mutagenesis and Related Subjects, 113, p.173-215), and was provided by MOLTOX™. It was stored at a temperature of less than −70° C. The microsomal fraction S9 was used in a concentration of 10% in S9-Mix. The protocol applied was as follows:
the following was introduced into 3 hemolysis tubes:
   assay without metabolic activation:
0.1 mL of the different concentrations of the test elements,
0.5 of sterile phosphate buffer, 0.2 M, pH 7.4,
2 mL of top agar for S. typhimurium,
0.1 mL of bacterial inoculum (TA100).
   assay with metabolic activation:
0.1 mL of the different concentrations of the test elements.
2 mL of top agar for S. typhimurium,
0.1 mL of bacterial inoculum (TA100),
0.5 mL of S9-Mix.
Mix and pour onto surface of bottom agar that has already been placed in Petri dishes.
Incubate at 37° C.±2° C. for 48 to 72 hours.
   These assays were carried out for each test: preliminary cytotoxicity test, test 1 and test 2. The untreated control, the negative controls and the positive controls produced during the pre-incubation method were incubated for 20 at 30 minutes at 37° C.±2° C. before pouring the top agar.
   The protocol that was applied was as follows:
For S. typhirnuriurn, introduce the following into 4 fractions of 2 mL of top agar:
   0.1 mL of phosphate buffer, 0.2 M, pH 7.4,
   0.1 mL of solvent,

### 0.1 mL of S9-Mix.

0.1 mL of the test element preparation at the highest concentration,
A fraction of 2 mL of top agar was used to control the sterility of S. typhimurium.
Mix and pour onto surface of bottom agar that has already been placed in Petri dishes.
Incubate at 37° C.±2° C. for 48 to 72 hours.
The test was carried out in triplicate.
No bacterial growth may be observed.
   A test without metabolic activation and a test with metabolic activation were carried out for at least 5 concentrations of the test extract.

### Expression of Results and Interpretation

A number of criteria can be used to determine whether a result is positive, in particular an increase in the number of revertants correlated with the dose of the item to be tested, or a reproducible increase in the number of revertants at one or more concentrations, with or without metabolic activation.
The test element is considered to be mutagenic if at the end of the verification steps, a dose-effect relationship has been obtained in a reproducible manner for one or more of the 5 strains with and/or without metabolic activation. Mutagenicity is only considered for a given concentration when the number of revertants is at least equal to double the spontaneous reversion ratio for the strains TA98, TA100 and TA102 (R≥2) and three times the spontaneous reversion ratio for the strains TA1535 and TA153 (R≥3).
The test element is considered to be non-mutagenic if, at the end of test 1 and test 2, the number of revertants is still less than double the spontaneous reversion ratio for all of the concentrations of the test element that has been tested for the strains TA98, TA100 and TA102 (R<2) and less than three times the spontaneous reversion ratio for the strains TA1535 and TA1537 (R<3), with and without metabolic activation, and provided that the absence of a mutagenic effect has been confirmed as not being linked to the toxicity of the test concentrations.
   The preliminary study did not demonstrate any cytotoxicity for the test element; as a result, this range of concentrations was used for genotoxicity test 1.

Because of the result obtained for test 1, it was decided to use the same dilution range for test 2. The analysis of the revertants shows that:
No cytotoxic effects were observed,
None of the concentrations of the test extract exhibited a ratio R of greater than or equal to at least double the spontaneous reversion ratio for TA98, TA100 and TA102 and three times the spontaneous reversion ratio for TA1535 and TA1537, with and without metabolic activation,
No responses to the dose were observed, irrespective of the test system or the test conditions.

In view of the results obtained during this study, the protein hydrolysate in accordance with Example 1 may be considered to have neither mutagenic nor pro-mutagenic activity.

### 2. In Vitro 3T3 NRU Phototoxicity Test

The principle of the test is the comparison of the cytotoxicity of the protein hydrolysate in accordance with Example 1 in the presence and in the absence of a non-cytotoxic dose of UVA, on cells under culture. The cytotoxicity was evaluated by determining the cell viability with the aid of a vital stain: neutral red, 24 h after treatment with the reference elements and the peregrina protein hydrolysate in accordance with the invention with or without irradiation with UVA. The cells used were mouse embryo fibroblasts from the Balb/c 3T3 clone 31 (ATCC-CCL163) line. The positive control was a solution of chlorpromazine (CAS number: 69-09-0). The negative control was a diluent for the test and reference extract (buffered saline solution+/−1% solvent). The peregrina protein hydrolysate was tested at 8 concentrations on at least four culture wells per concentration being studied, in the presence or absence of UVA. The fibroblasts were trypsinized and two 96-well culture plates were inoculated in an amount of 100 μL of a 2×10⁵ cells/mL (i.e. 2×10⁶ cells per well) cell suspension in complete culture medium. The inoculated plates were incubated in an oven for 24 hours at 37° C., 5% CO₂. At the end of the incubation, semi-confluence of the cell mat was verified. The dilutions were prepared just before depositing them onto the cells. The pH of the highest concentration was measured; it was comprised between 6.5 and 7.8. the culture medium was eliminated; each well had already been carefully rinsed with 150 μL of PBS maintained at ambient temperature before being treated with 100 μL of each extract dilution or reference dilution. The culture plates were incubated in the dark for 1 h±5 minutes at 37° C. and 5% CO₂. Irradiation was carried out with the aid of a BIO SUN solar irradiation system (Vilber Lourmat RMX3W). The BIO SUN is a system that controls UV irradiation with the aid of a programmable microprocessor. The system continuously monitors the emission of UV light. The irradiation stops automatically when the energy delivered is equal to the programmed energy. The spectral irradiation of the test device was measured in the wavelength range 250 to 700 nanometers with a calibrated spectroradiometer.

One of the 2 plates was irradiated with its cover at ambient temperature, and the other plate was protected from UVA and stored at ambient temperature for the irradiation period. After irradiation, the treatment medium was sucked off and the cells were rinsed. Next, 100 μL of complete culture medium was carefully added and the plates were incubated for 18 to 22 h at 37° C. and 5% CO₂. The next day, the cell viability (growth, morphology, integrity of the monolayer) was evaluated by observations using a phase contrast microscope. The culture medium was eliminated, each well had already been rinsed and maintained at ambient temperature before being treated with 100 μL of the staining solution. The plates were replaced in the incubator for 3 h under the same conditions. The staining solution was eliminated and the cells were rinsed; then the rinsing solution was removed and 150 μL of desorption solution was added to each well. The plates were agitated until the crystals had completely dissolved. The absorbances were measured at 450 nm.

### Validation of Test:

The sensitivity of the cells to UVA was monitored over all of the approximately 10 passes, by evaluation of their viability after exposure to increasing doses of irradiation. The cells were cultured to the density used in the test. They were irradiated the next day at a dose of 2.5 to 9 J/cm² and the cell viability was determined a day later with the aid of a NRU test. The cells satisfied the quality criteria if their viability after irradiation at 5 J/cm² UVA was 80% or more of the viability of the references kept in the dark; at the higher dose of 9 J/cm² UVA, the viability had to be at least 50% of that of the references kept in the dark.

### Results:

The negative control had an absorbance of 0.4 or higher. Chlorpromazine, the positive control, had a Cl50 comprised between 0.1 and 2 μg/mL in the presence of UVA and comprised between 7 and 90 μg/mL in the absence of UVA. These results enabled the test to be validated, The concentration of peregrina protein hydrolysate in accordance with Example 1 providing 50% cell death in the presence or absence of UVA could not be estimated. The mortality never reached 50%. The concentration of peregrina protein hydrolysate giving 50% cell viability in the presence or absence of UVA could not be estimated. The viability was always more than 50%.

Conclusion: Under the experimental conditions employed, the peregrina protein hydrolysate in accordance with the invention may be considered to be non-phototoxic,

### 3. Evaluation of Ocular Irritant Potential by the in Vitro Cytotoxicity Study in Accordance With the Neutral Red Leaching Method on the SIRC Cell Line

This in vitro study was based on an evaluation of the cytotoxicity of the peregrina protein hydrolysate by determining the concentration causing 50% cell death (IC₅₀) on a cellular monolayer with the aid of the neutral red leaching technique. The cells used were SRC, rabbit cornea fibroblasts (ATCC-CCL60) which were free from mycoplasma.

The peregrina protein hydrolysate was diluted with physiological serum to 25% and 50%. The fibroblasts were trypsinized and two 24-well culture plates were inoculated In an amount of 1 mL of a 2×10⁵ cells/mL cell suspension in complete culture medium, The inoculated plates were incubated in an oven overnight at 37° C. and 5% CO₂. At the end of incubation, confluence of the cell mat was verified. The staining solution was prepared in a concentration of 0.5 mg/mL in complete culture medium. The culture medium was eliminated; 1 mL of the staining solution was deposited into each well. The plates were replaced in the incubator at 37° C. and 5% CO₂ for 3 h+/−15 minutes. After this contact period, the staining solution was eliminated and replaced by 1 mL of complete culture medium per well. The plates were maintained at ambient temperature for at least 30 minutes in order to stabilize the system before contact with the extract or the reference, Each well was rinsed with 2 mL of PBS, maintained at ambient temperature, then 500 μL of each dilution of peregrina protein hydrolysate or reference was deposited in contact with the cell mat, The contact time was 60 seconds (30 seconds for the positive control). The treatment was carried out well by well, starting the clock at the moment of depositing the peregrina protein hydrolysate or the reference. The plate was agitated manually throughout the treatment period. After 55 seconds (or 25 seconds for the positive control), the dilution was sucked off. At exactly 60 or 30 seconds, 5 successive rinses were carried out (5×2 mL of PBS maintained at ambient temperature). The supernatant was sucked off after each rinse and after the last rinse, the wells were kept free from medium while awaiting the revealing phase. After the treatment of the culture plate was complete, 1 mL of the desorption solution was deposited into each well. The plate was agitated for approximately 15 minutes until homogeneous staining was obtained. The solutions obtained for each culture well were removed and divided into 2 wells of a 96-well plate, i.e. 150 μL/well.

### Results:

The concentration of the peregrina protein hydrolysate providing 50% cell death was evaluated at >50%. The percentage cell death at 50% peregrina protein hydrolysate was evaluated to be 17%.

Conclusion: Under the experimental conditions employed, the cytotoxicity of the peregrina protein hydrolysate in accordance with the invention could be classified as being: negligible cytotoxicity.

### 4. Evaluation of the Skin Compatibility of a peregrina Protein Hydrolysate in Accordance With the Invention After Single Application Under an Occlusive Dressing for 48 Hours Under Dermatological Control

The aim of this study was to evaluate the degree of skin compatibility of the peregrina protein hydrolysate by epicutaneous testing, carried out on the outer anterior surface of the arm for 48 hours; and in general to evaluate the capacity of the protein hydrolysate to maintain the skin in a good condition. 10 healthy male or female volunteers aged from 18 to 65 years who had neither dry skin nor sensitive skin and who were free from any dermatological lesions in the treatment zone could be included in the study. The skin compatibility of the peregrina protein hydrolysate, prepared in the form of a lotion with 5% peregrina protein hydrolysate in accordance with Example 1 and 95% of a propane diol/sorbitol mixture were evaluated 48 hours after initial application, 30 to 40 minutes after removing the dressing. The reactions of the skin (erythema and edema) were given a score of 0 to 3 in accordance with the following scales:

TABLE 34

| Score | Erythema (E) | Edema (Oe) |
|---|---|---|
| 0 | No erythema | No edema |
| 0.5 | Barely perceptible erythema, very | Palpable edema, barely |
| | faint pink coloration on a portion | perceptible |
| | of the patch zone | |
| 1 | Slight erythema, pink coloration | Palpable and visible edema |
| | over entire surface of patch | |
| 2 | Moderate erythema, clearly colored | Clear edema with or without |
| | over entire patch surface | papules or vesicles |
| 3 | Large erythema, intense coloration | Large edema diffusing beyond |
| | over entire patch zone | the patch zone with or without |
| | | papules or vesicles |

All of the skin reactions (blisters, papules, vesicles, dryness, desquamation, roughness, soap effect, etc.) were evaluated in accordance with the following scale and reported descriptively:
0: no reaction,
0.5: very slight
1: slight
2: moderate
3: major.

At the end of the study, a mean irritation index (M.I.I.) was calculated using the following formula:

M.I.I.=sum of skin reactions (E+Oe+blisters+papules+vesicles)/number of volunteers analyzed [Math. 4]

The M.I.I. obtained enabled the tested peregrina protein hydrolysate to be classified using the following scale:

| | |
|---|---|
| M.I.I. ≤ 0.20 | not irritating |
| 0.20 < M.I.I. ≤ 0.50 | slightly irritating |
| 0.50 < M.I.I. ≤ 2 | averagely irritating |
| 2 < M.I.I. ≤ 3 | highly irritating |

Results: The Mean Irritation Index (M.I.I.) of the peregrina protein hydrolysate is equal to: 0.
Conclusion: The peregrina protein hydrolysate may be considered to be non-irritating after 48 consecutive hours of application to 12 volunteers.

### General Conclusion From Tests

The results of the tests carried out above are conclusive, and demonstrate for the peregrina protein hydrolysate in accordance with Example 1:
1) The ocular and skin irritation tests are negative
2) The phototoxicity tests are negative
3) The mutagenicity tests are negative.
   The safety of the peregrina protein hydrolysate in accordance with the invention has been demonstrated and it is ideal for large scale dermatological use without restrictions as to target populations.

1. A method for obtaining a protein hydrolysate from Moringa peregrina seed cake, characterized in that it comprises the following steps, in which:
a) unshelled mature seeds are collected from ripe Moringa peregrina fruit and are dried in order to obtain an internal moisture content of less than 8%,
b) the dried seeds are pressed in a manner such as to separate the oil from the remainder of the seed in a manner such as to obtain the cake comprising less than 6% by weight of residual oil,
c) the cake obtained in step b) is ground,
d) the ground cake obtained in step c) is dispersed in an aqueous phase,
e) the aqueous dispersion obtained in step d) undergoes chemical proteolysis for a period of approximately 2 hours, at a pH of more than 13 and at a temperature comprised between 16° C. and 25° C.,
f) the proteolysis is neutralized in order to stabilize the protein hydrolysate obtained,
g) the protein hydrolysate is recovered by solid/liquid separation,
h) the protein hydrolysate is purified by ultrafiltration and/or nanofiltration carried out with a cutoff threshold comprised between 100 and 25000 Da, then optionally,
i) the protein hydrolysate obtained in step h) is lyophilized.

2. The method as claimed in claim 1, characterized in that the nanofiltration is carried out in a manner such as to separate 3 bands of the protein hydrolysate comprising a band P1 for which the molecular weight is less than 10000 Da, a band P2 for which the molecular weight is comprised between 10000 and 17000 Da and a band P3 for which the molecular weight is approximately 23000 Da.

3. The method as claimed in one of claims 1 and 2, characterized in that the nanofiltration step h) is carried out with a cutoff threshold comprised between 1500 Da and 5000 Da, preferably with a cutoff threshold comprised between 3000 Da and 4500 Da.

4. The method as claimed in claims 1 and 2, characterized in that the nanofiltration step h) is carried out with a cutoff threshold comprised between 10000 Da and 17000 Da.

5. The method as claimed in claims 1 and 2, characterized in that the nanofiltration step h) is carried out with a cutoff threshold comprised between 17000 Da and 25000 Da.

6. A protein hydrolysate from seed cake that has not been shelled and has been harvested from ripe Moringa peregrina fruit obtained by the method as claimed in one of claims 1 to 5.

7. A protein hydrolysate from seed cake that has not been shelled and has been harvested from ripe Moringa peregrina fruit, characterized in that it comprises a major fraction P1 of amino acid derivatives, amino acids, peptides and glycopeptides for which the molecular weight is comprised between 1500 Da and 5000 Da, a fraction P2 of approximately 20% for which the molecular weight is comprised between 10000 and 17000 Da and a fraction P3 of approximately 20% for which the molecular weight is approximately 23000 Da, in that it is obtained by chemical proteolysis at a pH of more than 13 for a period of approximately 2 hours at a temperature comprised between 16° C. and 25° C. and in that it is liquid and has a density of more than 1 and preferably about 1.1.

8. The protein hydrolysate as claimed in claim 7, characterized in that it comprises between 0.3% and 3% of volatile compounds, wherein 50% of these compounds, i.e. between 0.15% and 1.5% of the extract, is constituted by light nitrile compounds, principally with isobutyronitrile and methylbutanenitrile; wherein 5% to 10% of these compounds, i.e. between 0.015% and 0.3% of the extract, is constituted by isothiocyanate derivatives, principally with isopropyl isothiocyanate and isobutyl isothiocyanate; wherein 1% to 5% of these compounds, i.e. between 0.003% and 0.15%, is constituted by essential oil, principally with eucalyptol, menthol and benzaldehyde.

9. The protein hydrolysate as claimed in one of claims 7 and 8, characterized in that it comprises a dry matter content comprised between 10% and 15%, preferably about 12.5%, comprising between 1% and 6% of nitrogen-containing compounds, in particular volatile nitrile derivatives in a proportion of 0.5% to 1.5%, preferably about 0.8%, and 20 mg/liter of polyphenols.

10. The protein hydrolysate as claimed in one of claims 7 to 9, characterized in that it comprises the major fraction P1 for which the molecular weight is comprised between 1500 Da and 5000 Da.

11. The protein hydrolysate as claimed in one of claims 7 to 9, characterized in that it comprises the fraction P2 of approximately 20% for which the molecular weight is comprised between 10000 Da and 17000 Da.

12. The protein hydrolysate as claimed in one of claims 7 to 9, characterized in that it comprises the fraction P3 of approximately 20% for which the molecular weight is approximately 23000 Da.

13. The protein hydrolysate as claimed in one of claims 7 to 9, characterized in that it comprises the fraction P1 for which the molecular weight is comprised between 1500 Da and 5000 Da and the fraction P2 for which the molecular weight is comprised between 10000 and 17000 Da.

14. A protein hydrolysate from Moringa peregrina seed cake as claimed in claim 6 or claim 7, for its application as a drug.

15. A pharmaceutical or dermatological composition, characterized in that it comprises, as an active agent, an effective quantity of a protein hydrolysate from Moringa peregrina seed cake as claimed in one of claim 6 or 7, and a physiologically acceptable excipient.

16. The pharmaceutical composition as claimed in claim 15, characterized in that it is formulated for ingestion.

17. The dermatological composition as claimed in claim 15, characterized in that it is formulated for local application to the skin and the mucous membranes.

18. The composition as claimed in claims 15 to 17, characterized in that the protein hydrolysate of Moringa peregrina seed cake is present in the composition in a concentration of 0.0001% to 40% by weight with respect to the total weight of the composition.

19. A composition as claimed in one of claims 15 to 18, for its use as a drug for the treatment of fibrotic diseases and the treatment of inflammation, characterized in that it comprises, as the active agent, an effective quantity of the protein hydrolysate as claimed in claim 10 or 11, preferably as claimed in claim 10.

20. A composition as claimed in one of claims 15 to 18, for its use as a drug for the treatment of cancer, characterized in that it comprises, as the active agent, an effective quantity of the protein hydrolysate as claimed in claim 12.

21. A composition as claimed in one of claims 15 to 18, for its use as a drug for the treatment of infectious diseases of the bacterial or viral type, and in particular in order to inhibit spike COV2 proteins, characterized in that it comprises, as the active agent, an effective quantity of the protein hydrolysate as claimed in claim 6 or 7.

22. A composition as claimed in one of claims 15 to 18, for its use as a drug for the treatment of genetic drift and of pathologies associated with skin pigmentation, characterized in that it comprises, as the active agent, an effective quantity of the protein hydrolysate as claimed in claim 13.
